(19) United States
(12) Patent Application Publication
(10) Pub. No.: US 2026/0345320 A1
(43) Pub. Date: Nov. 5, 2026

(54) FRAME FOR PROSTHETIC HEART VALVE

(71) Applicant: Edwards Lifesciences Corporation, Irvine, CA (US)

(72) Inventors: Nikolai Gurovich, Hadera (IL); Karin Lavon, Ra'anana (IL); Michael Bukin, Pardes Hanna (IL); Tamir S. Levi, Zikhron Yaakov (IL)

(73) Assignee: Edwards Lifesciences Corporation, Irvine, CA (US)

(21) Appl. No.: 19/654,830

(22) Filed: Apr. 22, 2026

Related U.S. Application Data
(63) Continuation of application No. PCT/US2024/052495, filed on Oct. 23, 2024.
(60) Provisional application No. 63/592,864, filed on Oct. 24, 2023.

(51) Int. Cl. A61F 2/24

(52) U.S. Cl. CPC A61F 2/2418; A61F 2230/0006; A61F 2250/0036; A61F 2250/0037

(57) ABSTRACT

A prosthetic heart valve can include a radially expandable and compressible annular frame including a plurality of interconnected angled struts defining a plurality of circumferentially extending rows of cells arranged between an outflow end and an inflow end of the frame. The plurality of interconnected angled struts can be arranged to form a plurality of circumferentially extending rows of struts, including an outflow row of struts at the outflow end of the frame, one or more intermediate rows of struts, and an inflow row of struts at the inflow end of the frame, the one or more intermediate rows of struts disposed between the outflow row of struts and the inflow row of struts. Each strut of the outflow row of struts can have a width greater than a width of each strut of the one or more intermediate rows of struts.

### CROSS REFERENCE TO RELATED APPLICATIONS

This application is a continuation of International Patent Application No. PCT/US2024/052495, filed Oct. 23, 2024, which claims the benefit of U.S. Provisional Application No. 63/592,864, filed Oct. 24, 2023. The prior applications are incorporated herein by reference in their entirety.

### FIELD

The present application relates to expandable prosthetic heart valves, including frames for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, transcatheter heart valves comprise a radially expandable and compressible cylindrical metal frame and prosthetic leaflets mounted inside the frame. In some examples, a prosthetic heart valve can be implanted within the aortic root, which includes the right and left coronary ostia and is defined between the native aortic valve annulus and the sinotubular junction (STJ). The prosthetic heart valve can be implanted either within the native aortic valve or within a previously implanted prosthetic heart valve (e.g., previously implanted via a valve-in-valve (ViV) procedure). However, during such implantations, there can be a risk of at least partially blocking the coronary ostia either by native aortic valve leaflets that are pushed sideways during expansion of the prosthetic heart valve, by prosthetic leaflets of a previously implanted prosthetic heart valve that are similarly pushed sideways during expansion of a new prosthetic heart valve during a ViV procedure, and/or by the overlapping frames of the two valves following the ViV procedure. As a result, access to the coronary arteries for later interventions (such as with a catheter) may be made more difficult.

Accordingly, a need exists for improved frame designs for prosthetic heart valves.

### SUMMARY

Described herein are prosthetic heart valves, delivery apparatuses, and methods for implanting prosthetic heart valves. In particular, described herein are examples of radially expandable and compressible frames for prosthetic heart valves. The frame of a prosthetic heart valve can comprise a plurality of interconnected struts that define a plurality of rows of cells arranged between an outflow end and an inflow end of the frame.

In some examples, the frame can comprise reinforced or stiffened outflow and/or inflow end portions thus increasing the frame's radial resistive force, while preventing or mitigating the risk of leaflet damage, which typically occurs at or adjacent a midsection of the frame. The reinforced end portions can further facilitate synchronized deployment (i.e., the expansion of the outflow and inflow end portions of the frame at the same or substantially the same rate), thereby allowing the frame to assume a desired cylindrical configuration rather than an hourglass, or V-shaped configuration.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a plurality of circumferentially extending rows of struts, including an outflow row of struts at the outflow end of the frame, one or more intermediate rows of struts, and an inflow row of struts at the inflow end of the frame.

In some examples, each strut of the outflow row of struts has a width greater than a width of each strut of the one or more intermediate rows of struts.

In some examples, each strut of the inflow and outflow rows of struts can have the same width.

In some examples, the struts of the intermediate rows of struts can have widths that differ from row to row.

In some examples, each strut of the first row of struts has a first length, each strut of the second of struts has a second length, each strut of the third row of struts has a third length, and each strut of the fourth row of struts has a fourth length, and the first length, second length, third length, and fourth length can each be different from one another.

In some examples, each strut of the inflow row of struts has a width greater than the width of each strut of the one or more intermediate rows of struts and less than the width of each strut of the outflow row of struts.

In some examples, the prosthetic heart valve comprises a plurality of interconnected angled struts defining a plurality of circumferentially extending rows of cells arranged between an outflow end and an inflow end of the frame.

In some examples, the plurality of circumferentially extending rows of cells comprises four rows of cells.

In some examples, the plurality of circumferentially extending rows of cells comprises a first row of first cells disposed at the outflow end and at least partially defined by the first and second rows of struts, and a second row of second cells disposed adjacent to the first row of first cells and at least partially defined by the second and third rows of struts, wherein each first cell has a first axial length that is longer than a second axial length of each second cell.

In some examples, the outflow row of struts form pairs of angled struts, wherein the struts of each pair of angled struts are connected together at their adjacent ends by an outflow apex region, and wherein the outflow apex region curves between the pair of angled struts and has a narrowed width relative to each strut of the pair of angled struts.

In some examples, each row of struts can comprise struts having a different width than the struts of each other row of struts.

In some examples, the first and fourth rows of struts can comprise struts having the same width. In some examples, the second rows of struts can comprise struts having a different width than the struts in the third row of struts.

In some examples, each row of struts can comprise struts having a different length than the struts of each other row of struts.

In some examples, the first and fourth rows of struts can comprise struts having the same length. In some examples, the second rows of struts can comprise struts having a different length than the struts in the third row of struts.

In some examples, each row of struts can comprise angles between each pair of adjacent struts, and the angles can be different for each row of struts.

In some examples, the angles between struts of the first row can be the same as the angles between struts of the fourth row. In some examples, the angles between struts of the second row and struts of the third row can be different from one another.

In some examples, the prosthetic valve can further comprise a sealing member disposed on a radially outer facing surface of the frame. In some examples, the height of the sealing member is equal to a height of the inflow row of cells of the frame.

In some examples, the sealing member crosses the outflow row of cells at a selected height and the distance between two adjacent angled struts at that height is the sealing member gap. In some examples, the sealing member gap is narrow enough to prevent the sealing member from protruding radially through the outflow row of cells.

In some examples, the prosthetic valve can comprise a valvular structure disposed within the frame, the valvular structure comprising a plurality of leaflets. In some examples, the outflow edge portion of each leaflet is colinear with the outflow edges of laterally projecting tabs that extend from a main body of the leaflet.

In some examples, a prosthetic heart valve comprises one or more of the components recited in Examples 1-58 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic heart valve at an implantation site.

FIG. 5A is a side view of a portion of an exemplary frame for a prosthetic heart valve in a radially expanded configuration.

FIG. 5B is a side view of the portion of the frame of FIG. 5A in a radially compressed configuration.

FIG. 6 is a side view of the complete frame of FIG. 5A in a straightened (non-annular) state.

FIG. 7 is a side view of a portion of another exemplary frame for a prosthetic heart valve where angled struts of the frame assume an inwardly bent orientation in the radially compressed state of the frame.

FIG. 8 is a side view of a portion of another exemplary frame for a prosthetic heart valve where angled struts of the frame assume a relatively straight vertical orientation in the radially compressed state of the frame.

FIG. 9 is a side view of a portion of a frame for a prosthetic heart valve, according to one example.

FIG. 10 is a perspective view of the complete frame of FIG. 9 in a radially expanded configuration.

FIG. 11 is a side view of the complete frame of FIG. 9 is a straightened (non-annular) state.

FIG. 12 is a side view of a portion of the frame of FIG. 9, according to one example.

FIG. 13 is a side view of a portion of the frame of FIG. 9, according to one example.

FIG. 14 is a side view of a portion of an exemplary frame for a prosthetic heart valve.

FIG. 15 is a side view of a portion of an exemplary frame for a prosthetic heart valve.

FIG. 16 is a graph illustrating the deployed outer diameters of the frames illustrated in FIGS. 14-15 and 17 at different axial locations along the frame.

FIG. 17 is a side view of a portion of an exemplary frame for a prosthetic heart valve.

FIG. 18 is a side view of a portion of an exemplary frame for a prosthetic heart valve.

FIG. 19 is a side view of a portion of an exemplary frame for a prosthetic heart valve.

FIG. 20 is a side view of a portion of an exemplary frame for a prosthetic heart valve.

FIG. 21 is a cross-sectional view of a portion of the frame shown in FIG. 19 including a sealing member disposed on the frame.

FIG. 22 is a side view of a portion of the frame shown in FIG. 18 and a leaflet shown in a flattened configuration.

FIGS. 23A-23D are side views of a portion of an exemplary frame for a prosthetic heart valve shown at different working diameters.

FIG. 24 is a schematic view of a host valve and a guest valve in a valve-in-valve procedure.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can comprise a radially expandable and compressible annular frame and a plurality of leaflets attached to the frame. The frame can include a plurality of rows of cells formed by interconnected struts of the frame. The plurality of rows of cells can include a first row of cells arranged at an outflow end of the frame. In some examples, the cells of the first row of cells are elongated in an axial direction relative to cells of remaining rows of cells of the frame.

Additionally or alternatively, in some examples, in order to further increase a size of the cells of the first row of cells for increased coronary access following implantation, the cells of the first row of cell can be wider in a circumferential direction relative to cells of remaining rows of cells of the frame. For example, in some instances there may be one cell in the first row of cells for every two cells in each remaining row of cells (for example, due to the cells in the first row being twice as wide as the cells in the remaining rows of cells). As a result, cells of a second row of cells disposed adjacent to and connected to the first row of cells can include free apices that are unattached to (additional) struts defining the first row of cells.

Since the leaflets of the prosthetic valve move between a closed state and an open state during operation of the prosthetic valve (when implanted in a patient's body), they may contact (in their open state) these free apices of the second row of cells. In some instances, this can decrease a longevity of the leaflets.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

In some instances, when the prosthetic valve is in the radially compressed state and the angled struts of the frame assume a more vertical orientation, the leaflets can become caught between adjacent struts of the frame. This can also decrease a longevity of the leaflets.

Further described herein are various examples of frames for prosthetic heart valves comprising a first row of cells disposed at a first end of the frame (for example, an outflow end) that are wider in a circumferential direction than cells of remaining rows of cells of the frame, thereby resulting in exposed or free apices formed by a row of angled struts partially defining a second row of cells that is adjacent to the first row of cells. The free apices can have a first surface facing the outflow end of the frame and an opposing second surface facing the inflow end of the frame, where the first surface has a constant convex curvature extending between adjacent ends of a pair of angled struts of the row of angled struts. In some examples, the free apices or apex regions can have a narrowed width relative to the angled struts to which they are connected. As a result, these free apices or apex regions can be more atraumatic and may not interfere with the leaflets of the prosthetic heart valve as the leaflets open and close during operation of the prosthetic heart valve. As a result, a longevity of the leaflets can be increased.

In some instances, the stiffness of the frame of the prosthetic valve can be increased to withstand radial resistive and crushing forces, during radial expansion of the prosthetic heart valve the expansion member (e.g., a balloon of the delivery apparatus) can press the leaflets up against the stiffened struts of the frame, particularly when the prosthetic valve is deployed to relatively larger diameters (e.g., from about 29 mm to about 31 mm). This can also decrease a longevity of the leaflets.

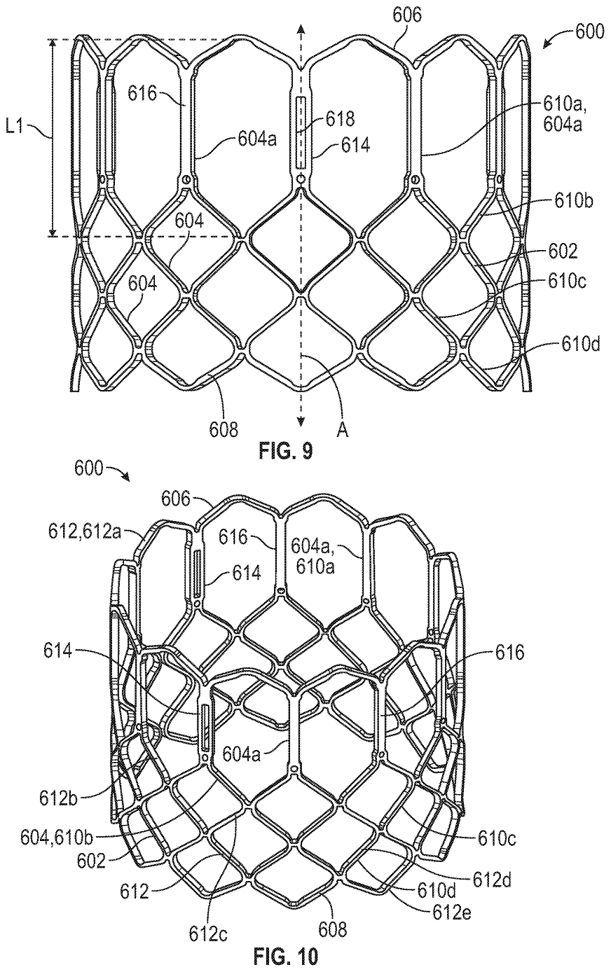

Described herein (see e.g., FIGS. 9-13 and the accompanying description thereof) are various examples of frames for prosthetic heart valves comprising thicker and/or wider struts at the outflow and/or inflow ends of the frame. Such frames have increased stiffness at the outflow and/or inflow ends (also referred to as “reinforced end portions”), thus increasing the frame's radial resistive force, while preventing or mitigating the risk of leaflet damage, which typically occurs at or adjacent a midsection of the frame. As a result, a longevity of the leaflets can be increased. The reinforced end portions can further facilitate synchronized deployment (i.e., the expansion of the outflow and inflow end portions of the frame at the same or substantially the same rate), thereby allowing the frame to assume a desired cylindrical configuration rather than an hourglass, or V-shaped configuration.

### Examples of the Disclosed Technology

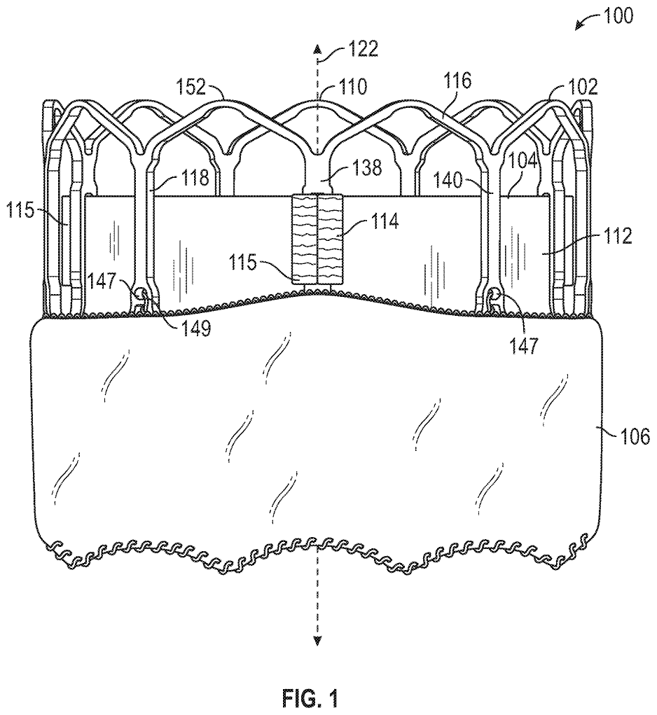

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Pat. No. 10,363,130, which is incorporated by reference herein. In another example, the disclosed prosthetic valves can be implanted within a docking device implanted within or at the native mitral valve, such as disclosed in US Publication No. 2022/0079749, which is incorporated herein by reference. In another example, the disclosed prosthetic valves can be implanted within a docking device implanted within the superior or inferior vena cava for replacing the function of a diseased tricuspid valve, such as disclosed in U.S. Pat. No. 11,291,540, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (e.g., by sutures). However, in alternate examples, the cusp edge portion of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in PCT Publication No. WO 2023/244612, which is incorporated by reference herein.

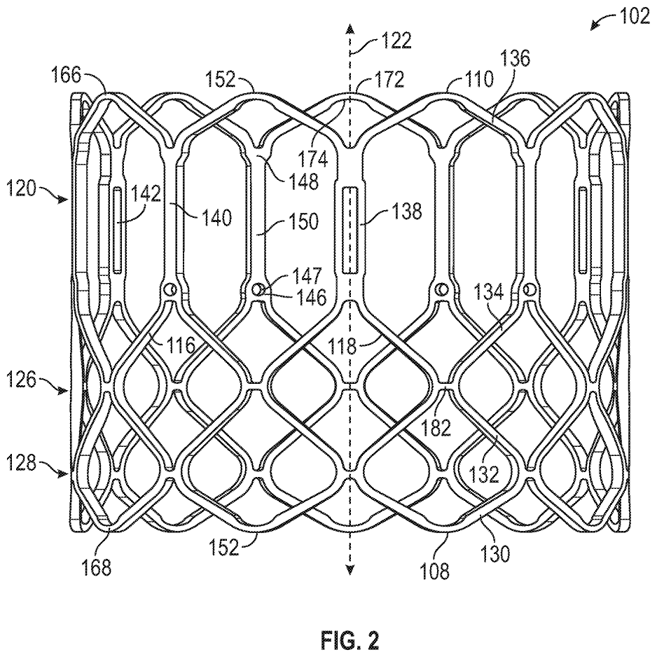

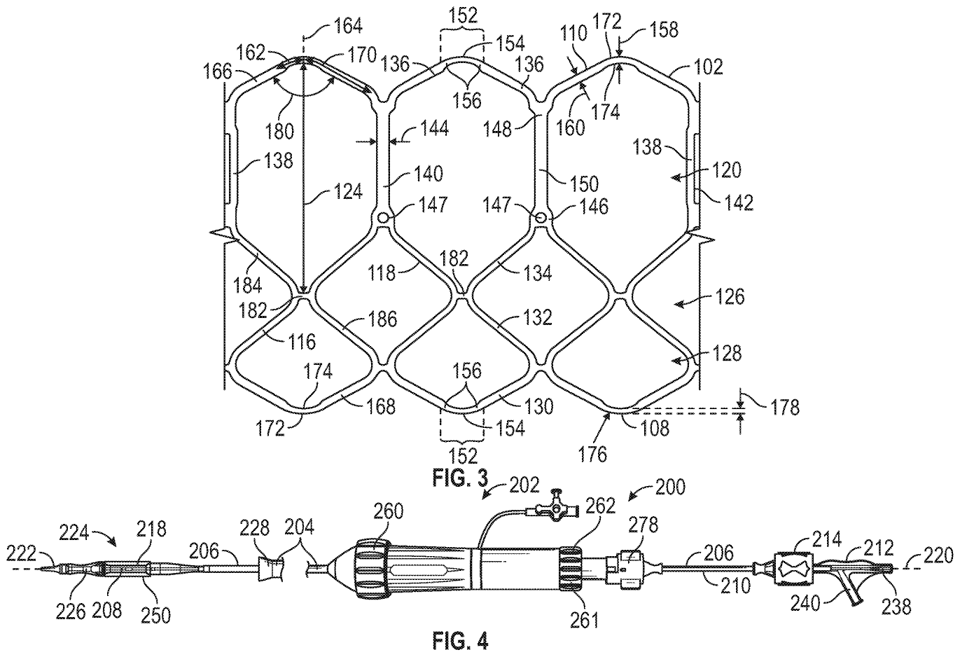

The frame 102 can be radially compressible and expandable between a radially compressed (or collapsed) configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (e.g., stainless steel, etc.) or self-expanding materials (e.g., Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (e.g., the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of open cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. See e.g., frame 600 in FIGS. 9-11. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (or axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIGS. 2 and 3) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (e.g., double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in PCT Publication WO2023/086548, which is incorporated by reference herein in its entirety.

As shown in FIGS. 2 and 3, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The interconnected struts 116 can also comprise horizontal struts 182 that extend between adjacent cells 118 of a row of cells of the frame 102 (FIGS. 2 and 3). The horizontal struts 182 can extend in a circumferential direction and also be referred to as circumferentially extending struts 182. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut A extending between an axially extending window strut 138 and the horizontal strut 182 and an angled strut 186 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts to follow a shape that more closely matches a shape of the scallop line of the leaflets when the frame 102 is in the radially expanded configuration (as shown in FIGS. 2 and 3). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (e.g., ±0.03 mm). In alternate examples, the first length 162 is in a range of 0.3-0.7 mm, 0.4-0.6 mm, 0.45-0.55 mm, or about 0.5 mm (e.g., ±0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant convex curvature).

As used herein, “constant convex curvature” can refer to a continuously curved surface which is convex and which does not have an inflection point (no change in direction of the curvature).

Each apex region 152 can have a radius of curvature 176, along the curved outer surface 172 (e.g., in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (e.g., the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 414, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a lar If ger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle F can be in a range of 120 (not inclusive) to 140 degrees (e.g., such that the angle 180 is greater than 120 degrees and less than or equal to 140 degrees).

Additional details and examples of frames for prosthetic heart valves that include apex regions can be found in PCT Publication No. WO2022/226147, which is incorporated by reference herein in its entirety.

FIG. 4 shows a delivery apparatus 200, according to an example, that can be used to implant an expandable prosthetic heart valve (e.g., the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 4), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 4, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 4) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Publication No. WO2022/046585 which is incorporated by reference herein in its entirety.

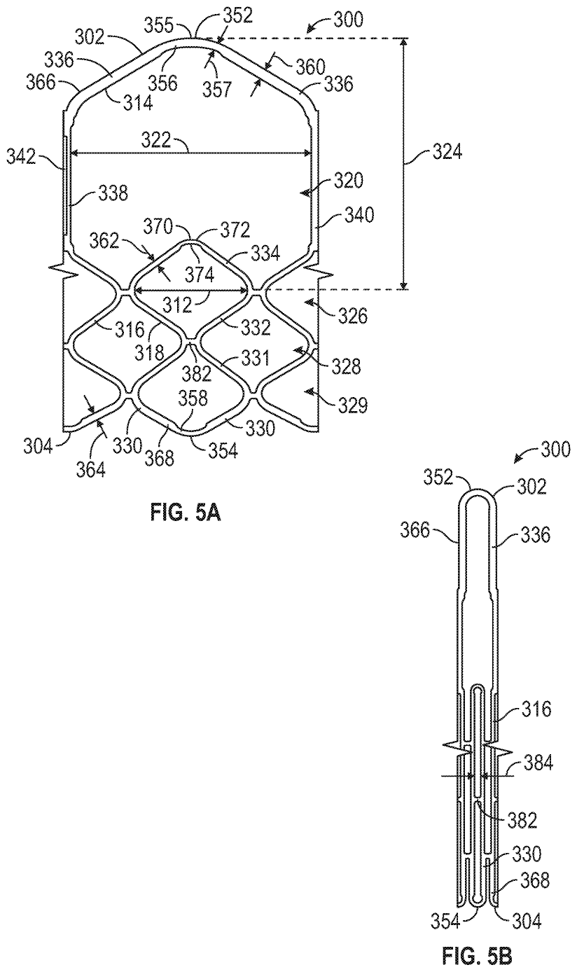

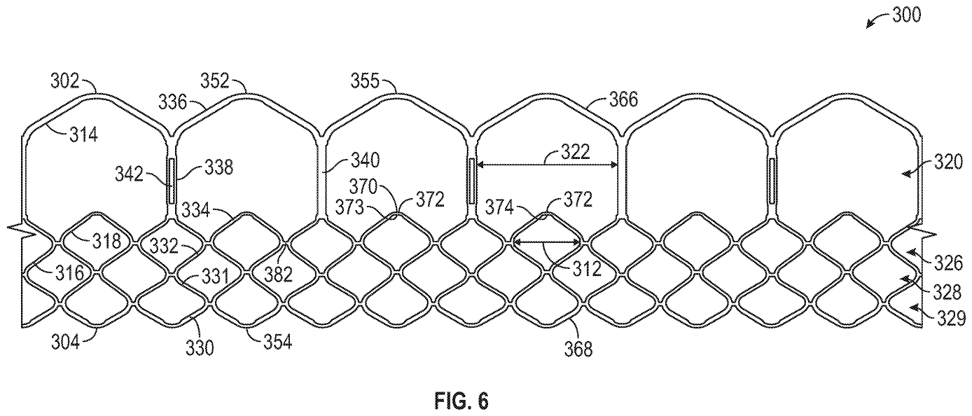

FIGS. 5A-6 illustrate another exemplary frame 300 for a prosthetic heart valve. In some examples, the frame 300 can be used in lieu of the frame 102 in the prosthetic heart valve 100 of FIG. 1. The frame 300 can be radially compressible and expandable between a radially compressed (or collapsed) configuration (FIG. 5B) and a radially expanded configuration (FIG. 5A). A portion of the frame 300 is shown in FIGS. 5A and 5B while an entirety of the frame 300 is shown in a straightened (non-annular) configuration in FIG. 8. It should be noted that the frame 300 can assume an annular configuration, such as that shown in FIG. 2.

The frame 300 can comprise any of the plastically-expandable materials (e.g., stainless steel, etc.) or self-expanding materials (e.g., nickel titanium alloy (NiTi), such as nitinol) materials discussed above with reference to the frame 102.

The frame 300 can comprise a plurality of interconnected struts 316 which form multiple rows of open cells 314, 318 between an outflow end 302 and an inflow end 304 of the frame 300. In some examples, as shown in FIGS. 5A-6, the frame 300 can comprise four rows of cells 314, 318 with a first (upper in the orientation shown in FIGS. 5A-6) row of cells 320 disposed at the outflow end 302. The first row of cells 320 comprises cells 314 that are elongated in an axial direction (relative to a central longitudinal axis of the frame 300 and a direction extending between the outflow end 302 and the inflow end 304) and wider in a circumferential direction, as compared to cells 318 in the remaining rows of cells. For example, the cells 314 of the first row of cells 320 can have a longer axial length 324 (FIG. 5A, as measured from an outflow end to an inflow end of the cell 320) and a larger width 322 than cells 318 in the remaining rows of cells. The increased width 322 and axial length 324 of the cells 314 of the first row can provide a larger opening for coronary access through the frame 300.

The remaining rows of cells can include a second row of cells 326, a third row of cells 328, and a fourth row of cells 329. The fourth row of cells 329 is disposed at the inflow end 304 and the second row of cells 326 is disposed between the first row of cells 320 and the third row of cells 328 (which is disposed adjacent to the fourth row of cells 329).

In some examples, as shown in FIG. 6, each of the second row of cells 326, the third row of cells 328, and the fourth row of cells 329 comprise 12 cells 318 and the first row of cells 320 comprises six cells 314. This arrangement of differing numbers of cells between the first row of cells 320 and the remaining rows of cells is due to the width 322 of the cells 314 of the first row of cells 320 being twice as wide as a width 312 of the cells 318 of the remaining rows of cells (for example, the adjacent second row of cells 326). Thus, in some examples, each cell 314 of the first row of cells 320 can span the width of two cells 314 (such as the third row of cells 328, as depicted in FIGS. 5A and 6). Said another way, the width 322 of each cell 314 can be double the width 312 of a cell 318.

In alternate examples, the frame 300 can comprise a different number of rows of cells, such as three (for example, similar to the frame 102 of FIGS. 2 and 3) or five. Additionally or alternatively, in some instances the frame 300 can include more or less than 12 and six cells per row (for example, ten and five cells per row). In some alternate examples, the cells 314 in the first row of cells 320 may not be elongated compared to cells 318 in the remaining rows of cells of the frame 300. In still other alternate examples, the width 322 of the cells 314 can be larger or smaller than shown in FIGS. 5A and 6 (such as being 1.5 times wider than the width 312 of the cells 318).

The interconnected struts 316 can include a plurality of angled struts 330, 331, 332, 334, and 336 arranged in a plurality of rows of circumferentially extending rows of angled struts, with the rows being arrayed along the length of the frame 300 between the outflow end 302 and the inflow end 304. For example, the frame 300 can comprise a first row of angled struts 330 arranged end-to-end and extending circumferentially at the inflow end 304 of the frame; a second row of circumferentially extending, angled struts 331; a third row of circumferentially extending, angled struts 332; a fourth row of circumferentially extending, angled struts 334, and a fifth row of circumferentially extending, angled struts 336 at the outflow end 302 of the frame 300. The circumferentially extending rows of angled struts (as well as additional components, such as the rows of cells) can be referred to herein as being “upstream” or “downstream” of other circumferentially extending rows of angled struts. As used herein, “upstream” or “downstream” are relative to the outflow end 302 of the frame (which is the downstream end of the frame) and the inflow end 304 of the frame (which is the upstream end of the frame) and a direction of blood flow through the frame 300 (from the inflow end 304 to the outflow end 302). For example, the fourth row of angled struts 334 is disposed upstream of the fifth row of angled struts 336.

The fifth row of angled struts 336 can be connected to the fourth row of angled struts 334 by a plurality of axially extending window struts 338 (which can be configured similar to the window struts 138 of frame 102, as described above) and a plurality of axial (or axially extending) struts 340 (which can be configured similar to the axial struts 140 of frame 102, as described above). The axially extending window struts 338 (which can also be referred to as axial struts that include a commissure window) define commissure windows (e.g., open windows) 342 that are spaced apart from one another around the frame 300, in a circumferential direction, and which are adapted to receive a pair of commissure tabs of a pair of adjacent leaflets arranged into a commissure (for example, commissure 114 shown in FIG. 1). In some examples, the commissure windows 342 and/or the axially extending window struts 338 defining the commissure windows 342 can be referred to herein as commissure features or commissure supports, each commissure feature or support configured to receive and/or be secured to a pair of commissure tabs of a pair of adjacent leaflets.

As shown in FIG. 6, one axial strut 340 can be positioned between, in the circumferential direction, two commissure windows 342 formed by the window struts 338. Each axial strut 340 and each window strut 338 extends from a location defined by the convergence of the lower ends (e.g., ends arranged inward of and farthest away from the outflow end 302) of two angled struts 336 (which can also be referred to as an upper strut junction or upper elongated strut junction) to another location defined by the convergence of the upper ends (e.g., ends arranged closer to the outflow end 302) of two angled struts 334 (which can also be referred to as a lower strut junction or lower elongate strut junction). Each axial strut 340 and each window strut 338 forms an axial side of two adjacent cells of the first row of cells 320.

In some examples, a width 360 of the angled struts 336 can be larger than a width of the struts of the remaining rows of struts (for example, to provide increased strength to the larger/wider cells 314). For example, the width 360 can be larger than a width 362 of the angled struts 334 (which, in some examples, can also be the width of the angled struts 331 and 332). Additionally, in some examples, a width 364 of the angled struts 330 can be larger than the width 362. However, in some examples, the width 360 of the angled struts 336 can still be larger than the width 364 of the angled struts 330.

In some examples, the frame 300 can further comprise a plurality of apex regions 352 formed at the outflow end 302 and plurality of apex regions 354 formed at the inflow end 304. Each apex region 354 can extend and form a junction between two angled struts 330 at the inflow end 304 of the frame 300. The apex regions 354 can be configured the same as or similarly to the apex regions 152, as described above (and thus are not redescribed here for the sake of brevity).

Each apex region 352 can extend and form a junction between two angled struts 336 at the outflow end 302 of the frame 300. The apex regions 352 can also be configured similarly to the apex regions 152, as described above. For example, each apex region 352 can comprise an apex 355 and two thinned (or narrowed) strut portions 356 (FIG. 5A) extending from either side of the apex 355 to a corresponding, wider, angled strut 336 (at the outflow end 302). The thinned strut portions 356 of the apex regions 352 can have a width 357 that is smaller than a width 360 of the angled struts 336 (FIG. 5A).

In some examples, the width 357 can be from about 0.06-0.15 mm smaller than the width 360 of the angled struts 336. For example, in some instances, the width 360 can be approximately 0.46 mm and the width 357 can be approximately 0.4 mm.

However, since the cells 314 are wider as compared to cells in remaining rows of the frame 300 (and as compared to the cells 118 of the frame 102), the angled struts 336 have a greater length than the angled struts 330. Thus, the length of thinned strut portions 356 of each apex region 352 can be longer than the thinned strut portions 358 of each apex region 354, for example (FIG. 5A). However, relative proportions between the thinned strut portion 356 of the apex regions 352 and the angled struts 336 can be similar or the same as those described above for the apex regions 152 of frame 102 (for example, a length of the thinned strut portions 356 can be at least 25% of the length of the angled strut 336)

Each apex region 352 and two corresponding angled struts 336 at the outflow end 302 can form an outflow strut 366 and each apex region 354 and two corresponding angled struts 330 at the inflow end 304 can form an inflow strut 368. Due to the increased width 322 of the cells 314 of the first row of cells 320, the outflow struts 366 are longer than the inflow struts 368.

A length of the apex region 352 (e.g., an arc length along the two thinned strut portions 356, similar to the length 162 shown in FIG. 3) is at least 25% of a length of the outflow strut 366. Similarly, a length of the apex region 354 is at least 25% of a length of the inflow strut 368.

An overall shape or curvature of the apex regions 352 and 354 can be similar to that of apex regions 152, as described above. Exemplary dimensions for the apex regions 352 and 354 are described below in reference to the free apex regions 370.

In this way, the apex regions 352 can have a relatively small axial height and not add much to the overall axial height of the radially expanded frame 300 (similar to as described above for the apex regions 152, and despite the longer length of the outflow struts 366). As such, the axial height of the radially expanded frame 300 can be smaller than alternate valve frames that have apices that are more pointed and/or have a larger axial height.

Due to the increased width 322 of the cells 314 of the first row of cells 320 relative to the width 312 of the cells 318 of the second row of cells 326, a portion of the second row of cells 320 can be formed by struts with exposed or free apex regions 370 that are unattached to additional struts of the adjacent row of cells (the first row of cells 320). As used herein, “free apex regions” can refer to apex regions that are not attached (directly attached) to any other struts, except for the angled struts which they curve and extend between (such as a pair of angled struts 334 and a corresponding apex region 370 disposed therebetween).

For example, the free apex regions 370 are not attached to any axial struts 340 or axially extending window struts 338. As shown in FIGS. 5A-6, every other cell 318 of the second row of cells 326 can be defined by struts having a free apex region 370. In alternate examples, more than one cell 318 defined by struts having a free apex region 370 can be disposed between cells 318 that are directly connected to an axial strut of the adjacent row of struts (for example, the axial struts 340 or axially extending window struts 338).

As introduced above, in some examples, the free apex regions 370 can interact with the leaflets of the prosthetic heart valve. For example, when the leaflets (such as the leaflets 112 of FIG. 1) are secured to the frame 300 as described herein, the free apex regions 370 can be disposed at a level (or axial height) of a portion of the leaflets that open and close during operation of the prosthetic heart valve. Thus, in some instances, when the leaflets are in an open state when implanted in a patient, the leaflets can contact the free apex regions 370.

Thus, it is beneficial for the free apex regions 370 to have a curved outer surface 372, similar to the apex regions 352 and 354. For example, as shown in FIGS. 5A-6, each free apex region 370 can have a similar overall shape to the apex regions 352 and 354, as described above, such as having a continuously curved outer surface 372 with a constant convex curvature that curves between the angled struts 334 to which it is connected (for example, with a radius of curvature in any of the ranges described herein for any of the curved apex regions. The outer surface 372 can face in a downstream direction, or toward the outflow end 302.

As used herein, “constant convex curvature” can refer to a continuously curved surface which is convex and which does not have an inflection point (no change in direction of the curvature).

Each free apex region 370 can extend and form a junction between two angled struts 334 at and can comprise an apex and two thinned (or narrowed) strut portions extending from either side of the apex to a corresponding, wider, angled strut 334 (similar to the apex regions 352, 354 and/or 152). An overall shape or curvature of the free apex regions 370 can be similar to that of apex regions 152, as described above.

The free apex regions 370 (e.g., the thinned strut portions and apex of the free apex regions 370) have a narrowed width relative to the angled struts 334 to which they are connected, thereby forming an inner depression 374 on a cell side of each free apex region 370 (for example, the inner depression 374 is formed on an opposing inner surface 373 of the free apex region 370 that faces an upstream direction, or toward the inflow end 304). The width of the free apex regions 370 can be defined between the outer surface 372 and the inner surface 373.

The width of the free apex regions 370 can be from about 0.06-0.15 mm smaller than the width of the angled struts 334. For example, in some instances, the width of the free apex regions 370 can be approximately 0.18 mm and the width of the angled struts 334 can be approximately 0.24 mm.

A length of each free apex region 370 (e.g., an arc length along the apex regions 370, similar to the length 162 shown in FIG. 3) is at least 25% a length of the entire strut it is part of, where the strut is defined the include the free apex region 370 and the two angled struts 335 to which it is connected.

In some instances, the length of the free apex regions 370, the outflow apex regions 352, and/or the inflow apex regions 354 can be in a range of 0.5-4.7 mm or 0.9-3.7 mm (e.g., with the free apex regions 370 being on the lower end of the range and the outflow apex regions 352 being on the higher end of the range).

In some examples, a radius of curvature of the free apex regions 370, the outflow apex regions 352, and/or the inflow apex regions 354 can be in a range of 0.3-10 mm, 0.5-8 mm, or 0.2-20 mm.

In some instances, a ratio between the width of any of the apex regions 370, 352, and 354 and a width of the angled struts to which they connect (e.g., a ratio between the width of the free apex regions 370 and the width of the angled struts 334 and/or a ratio between the width 357 and the width 360 of the apex regions 352) can be in a range of 0.15-0.98, 0.4-0.8, or 0.6-0.9.

In some examples, a central longitudinal axis of each free apex region 370 can be aligned (overlapping) with a central longitudinal axis of a corresponding apex region 352. In alternate examples, the free apex regions 370 may not be aligned with corresponding apex regions 352 (for example, if the cells 314 are not twice as wide as the cells 318 in the second row of cells 326).

In this way, the free apex regions 370 can have a larger curved (less angled) outer surface that is more atraumatic and may not interfere with the leaflets of the prosthetic heart valve as the leaflets open and close during operation of the prosthetic heart valve. Thus, a long term durability of the prosthetic valve leaflets can be increased.

In alternative examples, the free apex regions 370 of the frame 300 may not include the curved outer surface 372 with the constant convex curvature. Instead, the free apex regions 370 can have an alternative shape that is similar to the junction between two angled struts 334 that connect to an axial strut 340 or axially extending window struts 338.

As shown in FIGS. 5A-6, the interconnected struts 316 of the frame 300 can also comprise horizontal struts 382 (which can be similar to horizontal struts 182 of frame 102, as described above) that extend between adjacent cells 318 of a row of cells of the frame 300. The horizontal struts 382 can connect angled struts of two adjacent rows of angled struts of the frame 300 to one another. As described above with reference to the horizontal struts 182 of the frame 102, the horizontal struts 382 can allow the angled struts 330, 331, 332, and 334 to follow a shape that closely matches a shape of the scallop line of the leaflets of the prosthetic valve when the frame 300 is in the radially expanded configuration (FIG. 5A). Additionally, the horizontal struts 382 can serve as spacers that maintain a specified gap 384 between the angled struts 330, 331, 332, and 334 when the frame 300 is in the radially compressed configuration (FIG. 5B), where the specified gap 384 can be minimized as much as possible (to minimize a crimp profile of the prosthetic valve) while still being large enough to reduce a risk of catching or pinching the leaflets of the prosthetic valve between the adjacent angled struts 330, 331, 332, and 334 when the frame 300 is radially compressed (or crimped).

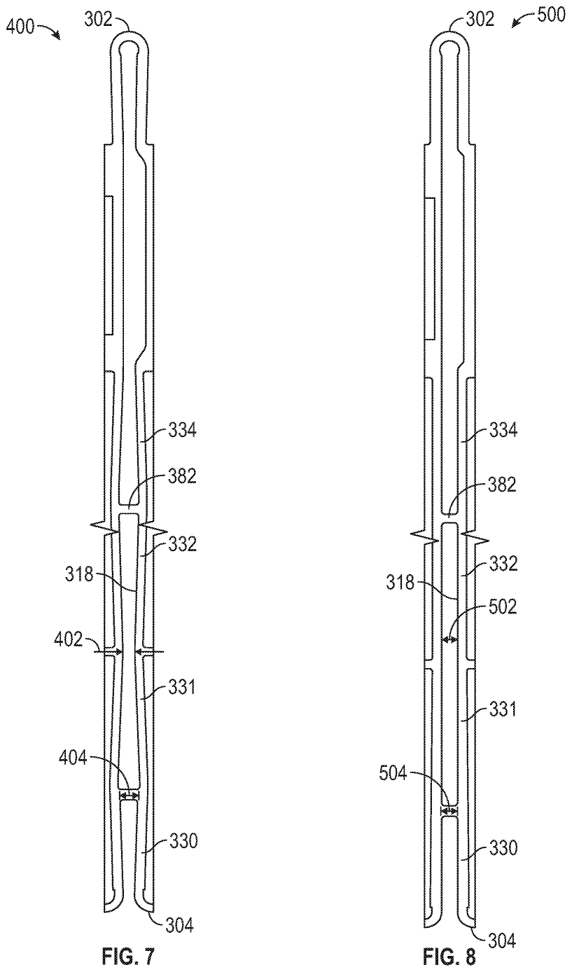

FIGS. 7 and 8 illustrate two examples of prosthetic valve frames 400 and 500, respectively, including horizontal struts 382 and angled struts 330, 331, 332, and 334 that assume either a relatively straight vertical orientation (FIG. 8) or an inwardly bent orientation (FIG. 7) when the frame is radially compressed (or crimped). The frames 400 and 500 of FIGS. 7 and 8, respectively, can be similar to the frame 300, but without the wider outflow cells (cells 314). However, it should be noted that the angling or straight orientation of the angled struts 330, 331, 332, and 334, as described below with reference to FIGS. 7 and 8, can also be applied to the frame 300.

When the angled struts 330, 331, 332, and 334 assume the inwardly bent (or angled) orientation in the compressed state of the frame 400 (FIG. 7), a length 404 of the horizontal struts 382 can be specified to retain a minimal gap 402 between the angled struts, as measured as the smallest width value (in the circumferential direction of the frame 400) between adjacent horizontal struts 382, for example. The minimal gap 402 can reduce a crimp profile of the frame 400 while avoiding pinching leaflets between the angled struts in the crimped state.

When the angled struts 330, 331, 332, and 334 assume the relatively straight vertical orientation (in the axial direction) in the compressed state of the frame 500 (FIG. 8), a length 504 of the horizontal struts 382 can be specified to retain a minimal gap 502 between the angled struts. The minimal gap 502 can reduce a crimp profile of the frame 500 while avoiding pinching leaflets between the angled struts in the crimped state.

In some examples, the horizontal struts 382 are dimensioned to retain the minimal gap 402 or 502 within a range of about 0.2 mm-0.7 mm or about 0.3 mm-0.4 mm.

In other examples, the minimal gap 402 or 502 in the crimped configuration can be a function of the prosthetic valve leaflet's thickness, and can account for compressibility of the tissue of the leaflets. For example, for a leaflet having a thickness of about 0.2 mm, a minimal gap 402 or 502 can be designed to accommodate a leaflet folded over itself, which may require a gap of at least 0.4 mm. However, if the tissue has about 50% compressibility, it may be sufficient to design for a minimal gap of about 0.2 mm.

In this way, a frame of a prosthetic heart valve can be configured to increase a durability and long term longevity of the prosthetic heart valve (for example, when implanted), while also reducing a crimp profile of the prosthetic heart valve and reducing stresses on the frame struts (for example, due to a shape of the apex regions).

FIGS. 9-11 illustrate another exemplary frame 600 for a prosthetic heart valve. In some examples, the frame 600 can be used in lieu of the frame 102 in the prosthetic heart valve 100 of FIG. 1. Frame 600 can comprise a greater number of circumferentially extending rows of cells (e.g., four rows), with a greater number of cells (e.g., twelve cells) in each row than frame 102. A prosthetic valve implementing frame 600 can be implanted using any of the methods, techniques, and/or delivery apparatuses described herein.

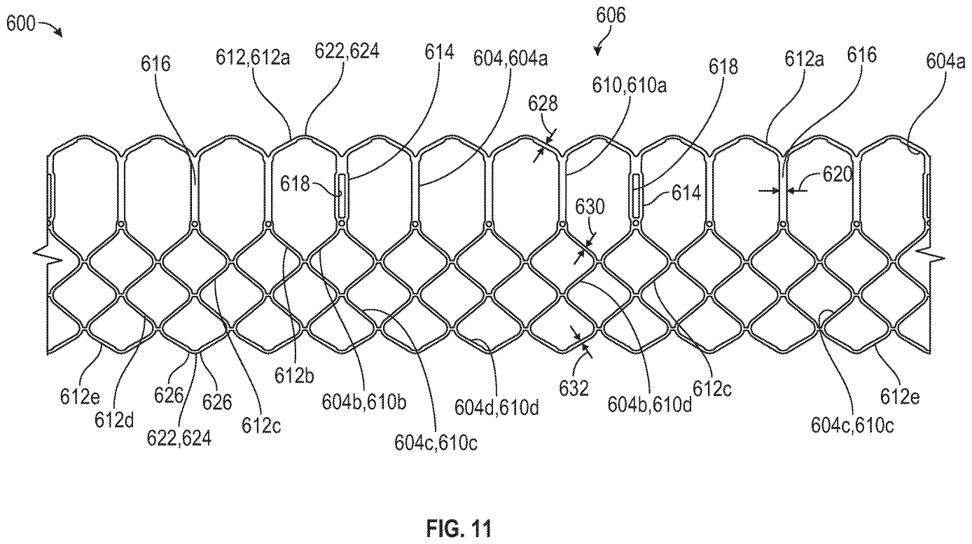

The frame 600 can be radially compressible and expandable between a radially compressed (or collapsed) configuration and a radially expanded configuration (the expanded configuration is shown in FIG. 10). FIGS. 9-11 show the frame alone, FIG. 9 shows a portion of the frame (though it should be understood that the frame is an annular structure such as shown in FIG. 10), and FIG. 11 shows a portion of the frame 600 is a straightened (non-annular) configuration (though it should be understood that the frame 60 can assume an annular configuration such as shown in FIG. 10).

The frame 600 can be made of any of various suitable plastically-expandable materials (e.g., stainless steel, etc.) or self-expanding materials (e.g., Nitinol). When constructed of a plastically-expandable material, the frame 600 (and thus the prosthetic valve including frame 600) can be crimped to a radially compressed state on a delivery catheter and then expanded inside a patient by an inflatable balloon or equivalent expansion mechanism. When constructed of a self-expandable material, the frame 600 (and thus the valve) can be crimped to a radially compressed state and restrained in the compressed state by insertion into a sheath or equivalent mechanism of a delivery catheter. Once inside the body, the valve can be advanced from the delivery sheath, which allows the valve to expand to its functional size. Suitable plastically-expandable materials used to form the frame are described above with reference to frame 102.

As shown in FIGS. 9-11, the frame 600 can comprise a plurality of interconnected struts 602 that form open cells 604 disposed in multiple rows 610 between the outflow end 606 and the inflow end 608. In some examples, such as shown in FIGS. 9-11, the frame 600 can comprise four circumferentially extending rows of cells 610 with a first (upper in the orientation shown in FIGS. 9-11) row of cells 610a disposed at the outflow end 606. The first row of cells 610a comprises cells 604a that are elongated in an axial direction (relative to a central longitudinal axis of the frame), compared to cells 604 in the remaining rows of cells 610. For example, cells 604a in the first row of cells 610a have a longer axial length L₁ than cells 604 in the remaining rows of cells 610, which can include a second row of cells 610b, a third row of cells, 610c, and a fourth row of cells 610d.

In some examples, such as shown in FIGS. 9-11, each row of cells 610 comprises twelve cells 604. Thus, the frame 600 can be referred to as a twelve-cell frame. In other examples, the frame 600 can have a greater or fewer number of circumferentially extending rows of cells 610 and/or a greater or fewer number of cells 604 in each row. In some examples, the cells 604a of the first row 610a may not be elongated relative to the other rows and/or the cells of the other rows (e.g., the second, third, or fourth rows 610b, 610c, 610d) may be elongated relative to the remaining rows.

The interconnected struts 602 can include a plurality of angled struts 612 arranged in a plurality of rows of circumferentially extending rows of angled struts, with the rows being arrayed along the length of the frame 600 between the outflow end 606 and the inflow end 608. For example, the frame 600 can comprise a first row of angled struts 612a arranged end-to-end and extending circumferentially at the outflow end 606 of the frame (referred to as the “outflow angled struts” 612a); a second row of circumferentially extending, angled struts 612b; a third row of circumferentially extending, angled struts 612c; a fourth row of circumferentially extending, angled struts 612d; and a fifth row of circumferentially extending, angled struts 612e at the inflow end 608 of the frame 600 (referred to as the “inflow angled struts” 612e). The second through fourth rows of angled struts can be referred to as the “intermediate angled struts” 612b-612d).

The first row of angled struts 612a can be connected to the second row of angled struts 612b by a plurality of axially extending window struts 614 (or window strut portions) and a plurality of axial (or axially extending) struts 616. The axially extending window struts 614 (which can also be referred to as axial struts that include a commissure window) define commissure windows (e.g., open windows) 618 that are spaced apart from one another around the frame 600, in a circumferential direction, and which are adapted to receive a pair of commissure tabs of a pair of adjacent leaflets arranged into a commissure. In some examples, the commissure windows 618 and/or the axially extending window struts 614 defining the commissure windows 618 can be referred to herein as commissure features or commissure supports, each commissure feature or support configured to receive and/or be secured to a pair of commissure tabs of a pair of adjacent leaflets.

One or more (for example, three, as shown in FIGS. 10 and 11) axial struts 616 can be positioned between, in the circumferential direction, two commissure windows 618 formed by the window struts 614. Each axial strut 616 and each window strut 614 extends from a location defined by the convergence of the lower ends (e.g., ends arranged inward of and farthest away from the outflow end 606) of two angled struts 612 (which can also be referred to as an upper strut junction or upper elongated strut junction) to another location defined by the convergence of the upper ends (e.g., ends arranged closer to the outflow end 606) of two angled struts 612 (which can also be referred to as a lower strut junction or lower elongate strut junction). Each axial strut 616 and each window strut 614 forms an axial side of two adjacent cells of the first row of cells 610a.

In some examples, as shown in FIG. 11, each axial strut 616 can have a width 620 (FIG. 11) that is larger than a width of the angled struts 612. As used herein, a “width” of a strut is measured between opposing locations on opposing surfaces of a strut that extend between the radially facing inner and outer surfaces of the strut (relative to a central longitudinal axis A (FIG. 9) of the frame 600). A “thickness” of a strut is measured between opposing locations on the radially facing inner and outer surfaces of a strut and is perpendicular to the width of the strut. In some examples, the width 620 of the axial struts 616 is 50-200%, 75-150%, or at least 100% larger than (e.g., double) the width of the angled struts 612 of the frame 600.

By providing the axial struts 616 with the width 620 that is greater than the width of other, angled struts 612 of the frame 600, a larger contact area is provided for when the leaflets (see e.g., leaflets 112 of valvular structure 104 described previously) contact the wider axial struts 616 during systole, thereby distributing the stress and reducing the extent to which the leaflets may fold over the axial struts 616, radially outward through the cells 604a. As a result, a long-term durability of the leaflets can be increased.

As described previously with reference to FIG. 1, the valvular structure can comprise one or more commissures (see e.g., commissures 114). Each commissure can extend through a commissure window 618 of the frame 600. Each commissure can be secured to the window struts 614 forming the commissure window 618. The cusp edge portion of each leaflet of the valvular structure can be secured to the frame 600 via one or more fasteners (e.g., sutures), such as described above with reference to prosthetic valve 100.

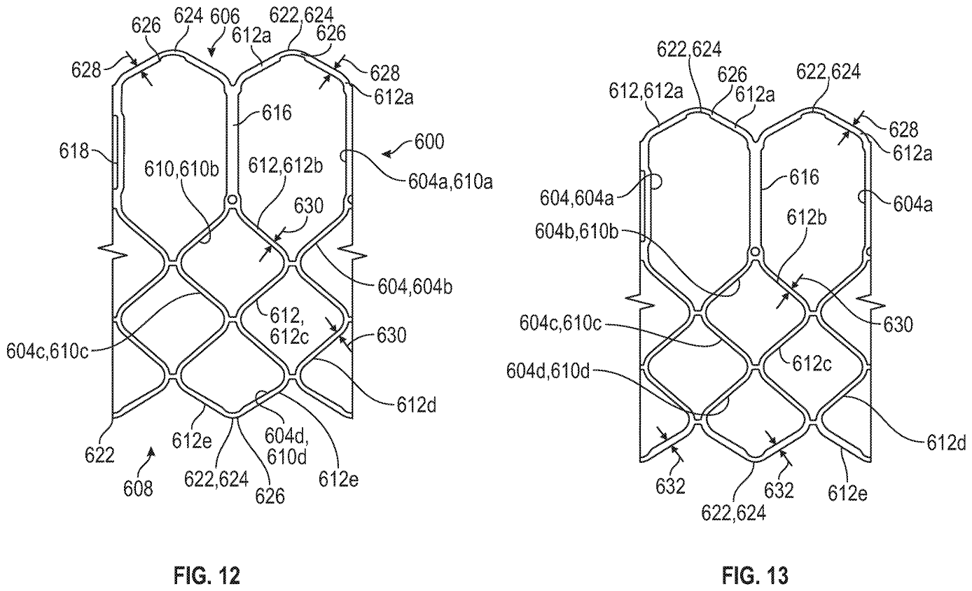

In some examples, the stiffness of the frame 600 can be increased to withstand radial resistive and crushing forces and to increase the longevity of the valvular structure, particularly when the prosthetic valve is deployed to larger diameters (e.g., from about 29 mm to about 31 mm). For example, referring to FIG. 12, in some examples, the angled struts 612a at the outflow end 606 of the frame 600 can be thicker and/or wider than the remaining angled struts 612b-612e.

As shown, the frame 600 can comprise a plurality of apex regions 622 formed at the inflow and outflow ends 606, 608 of the frame 600. Each apex region 622 extends and forms a junction between two angled struts 612e at the inflow end 608 or two angled struts 612a at the outflow end 606. As such, the apex regions 622 are spaced apart from one another around a circumference of the frame 600 at the inflow end 608 and the outflow end 606.

Each apex region 622 can comprise an apex 624 (the highest or most outward extending, in an axial direction, point) and two thinned (or narrowed) strut portions 626, one thinned strut portion 626 extending from either side of the apex 624 to a corresponding, wider, angled strut 612a (at the outflow end 606) or angled strut 612e (at the inflow end 608). In this way, each of the apex regions 622 at the outflow end 606 can form a narrowed transition region between and relative to the two angled struts 612a extending from the corresponding apex region 622 and each of the apex regions 622 at the inflow end 608 can form a narrowed transition region between and relative to the two angled struts 612e extending from the corresponding apex region 622.

In some examples, such as shown in FIG. 12, each of the outflow angled struts 612a can have a width 628 greater than a width 630 of the intermediate angled struts 612b-612d. For example, the outflow angled struts 612a can have a width 628 between about 0.32 mm and about 0.44 mm, between about 0.36 mm and about 0.42 mm, between about 0.39 mm and about 0.41 mm. In some examples, the width 628 of the outflow angled struts 612a can be about 0.4 mm. The intermediate angled struts 612b-612d can have a width 630 between about 0.22 mm and about 0.34 mm, between about 0.24 mm and about 0.32 mm, between about 0.26 mm and about 0.3 mm. In some examples, the width 630 of the intermediate struts 612b-612d can be about 0.28 mm.

The width 628 of the outflow angled struts 612a increases the stiffness of the outflow end 606 of the frame 600. Such a configuration advantageously provides greater radial resistive force and crush resistance at the outflow end 606 of the prosthetic valve 600 (which in some prosthetic valves is more prone to fatigue/failure), while the intermediate angled struts 612b-612d remain thinner (and thus less stiff). The less stiff intermediate angled struts 612b-612d can prevent or mitigate leaflet tearing during balloon expansion (as could potentially occur if the entire frame 600 were stiffer). For example, balloon expansion of some prosthetic valves can result in tearing of the leaflets if the expanding balloon presses them against an overly stiff mid-portion of the frame, particularly in the case of large diameter valves. Stiffening the outflow angled struts 612a while retaining the less stiff intermediate angled struts 612b-612d advantageously provides the desired radial resistance/crush resistance at the outflow end 606 without decreasing the longevity of the leaflets.

In some examples, the inflow angled struts 612e can also have increased stiffness relative to the intermediate angled struts 612b-612d. In some examples, the inflow angled struts 612e can have a width 632 greater than the width 630 of the intermediate angled struts 612b-612d, but less than the width 628 of the outflow angled struts 612a. For example, the inflow angled struts 612e can have a width between about 0.3 mm and about 0.42 mm, between about 0.32 mm and about 0.4 mm, between about 0.34 mm and about 0.38 mm. In some examples, the inflow angled struts 612e can have a width 632 of about 0.37 mm. In some examples, the inflow angled struts 612e can have the same width as the intermediate angled struts 612b-612d.

In other examples, the inflow angled struts 612e can have a width 632 that is equal to or greater than the width 628 of the outflow angled struts 612a. For example, both the inflow angled struts 612e and the outflow angled struts 612a could have widths 632, 628 of about 0.4 mm.

Increasing the stiffness of the inflow angled struts 612e further increases the overall stiffness of the frame 600 without specifically stiffening the intermediate angled struts 612b-612d, which is advantageous for the reasons described above.

Another advantage provided by the stiffened outflow angled struts 612a and/or inflow angled struts 612e is that they can facilitate synchronized deployment (e.g., the expansion of the outflow and inflow end portions of the frame 600 at the same or substantially the same rate), thereby allowing the frame 600 to assume a uniform cylindrical expansion profile. This can prevent an undesired hourglass or V-shaped frame configuration.

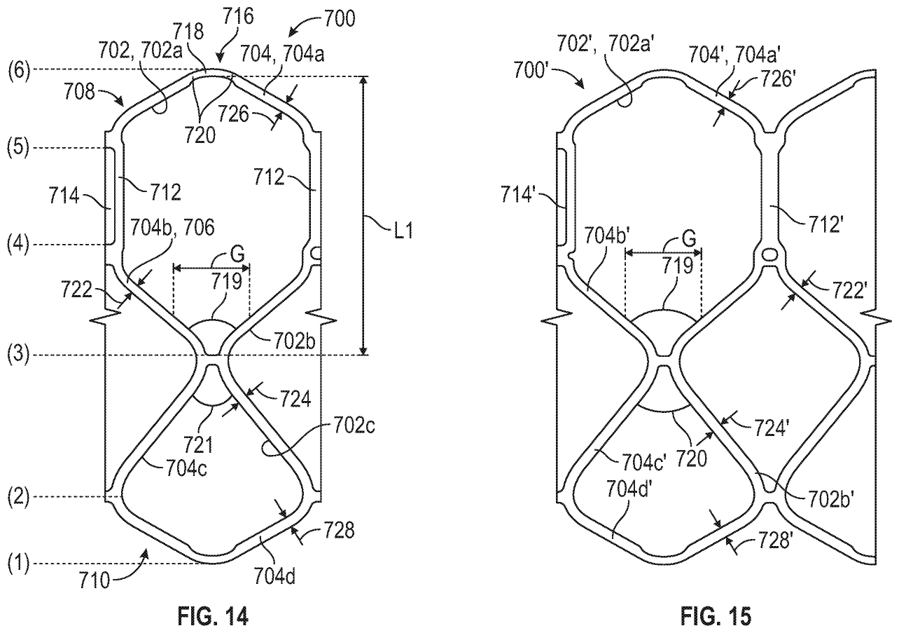

FIG. 14 illustrates another exemplary frame 700 for a prosthetic heart valve. In some examples, the frame 700 can be used in lieu of the frame 102 in the prosthetic heart valve 100 of FIG. 1 described previously. Frame 700 can be similar to frame 102, e.g., having three circumferentially extending rows of cells 702 and having nine cells in each row. A prosthetic valve implementing frame 700 can be implanted using any of the methods, techniques, and/or delivery apparatuses described herein.

The frame 700 can be radially compressible and expandable between a radially compressed (or collapsed) configuration and a radially expanded configuration. FIG. 14 shows a portion of the frame 700 (though it should be understood that the frame is an annular structure similar to frame 102 shown in FIG. 2). The frame 700 can be made of any of various suitable plastically-expandable materials (e.g., stainless steel, etc.) or self-expanding materials (e.g., Nitinol). Suitable plastically-expandable materials used to form the frame are described above with reference to frame 102.

As shown in FIG. 14, the frame 700 can comprise a plurality of interconnected struts 704 that form open cells 702 disposed in multiple rows 706 between the outflow end 708 and the inflow end 710. In some examples, such as shown in FIG. 14, the frame 700 can comprise three circumferentially extending rows of cells 702 with a first (upper in the orientation shown in FIG. 14) row of cells 702a disposed at the outflow end 708. The first row of cells 702a comprises cells 702a that are elongated in an axial direction (relative to a central longitudinal axis of the frame), compared to cells 702b, 702c in the remaining rows. For example, cells 702a in the first row of cells have a longer axial length L1 than cells in the remaining rows of cells, which can include a second row of cells 702b and a third row of cells, 702c.

The first row of angled struts 704a can be connected to the second row of angled struts 704b by a plurality of axially extending struts 712, some of which include commissure windows 714. In some examples, the commissure windows 714 and/or the axially extending window struts 712 defining the commissure windows 714 can be referred to herein as commissure features or commissure supports, each commissure feature or support configured to receive and/or be secured to a pair of commissure tabs of a pair of adjacent leaflets. The axially extending struts 712 and commissure windows 714 can be similar to axially extending struts 140 and commissure windows 142 described previously, except where indicated otherwise.

As described previously with reference to FIG. 1, the valvular structure can comprise one or more commissures (see e.g., commissures 114). Each commissure can extend through a commissure window 714 of the frame 700. Each commissure can be secured to the axial struts 712 defining the commissure window 714. The cusp edge portion of each leaflet of the valvular structure can be secured to the frame 700 via one or more fasteners (e.g., sutures), such as described above with reference to prosthetic valve 100.

In some examples, the frame 700 can comprise a plurality of apex regions 716 similar to apex regions 152 described previously and including an apex 718 and two thinned or narrowed strut portions 720 each extending from one side of the apex 718 to a corresponding angled strut 704a (at the outflow end 708) or angled strut 704d (at the inflow end 710). The thinned struts 720 and apex 718 together can be referred to as the apex region 716 or narrowed transition region between and relative to the two angled struts 704 extending from the apex region.

In some examples, the frame 700 can include inflow angled struts 704d and/or outflow angled struts 704a that are wider and/or thicker than the remaining angled struts 704b-704c. The remaining angled struts 704b-704c (i.e., the angled struts between the inflow angled struts 704d and outflow angled struts 704a) can also be referred to as the “intermediate struts.” Each angled strut 704b can have a width 722, and each angled strut 704c can have a width 724. In some examples, the width 722 and the width 724 can be the same. In other examples, such as shown in FIG. 14, the width 722 and the width 724 can be different from one another.

For example, intermediate angled struts 704b can have a width 722 less than the width 724 of the intermediate angled struts 704c. For example, the width 722 of the angled struts 704b can be between about 0.22 mm and about 0.43 mm, between about 0.24 mm and about 0.41 mm, between about 0.26 mm and about 0.39 mm, between about 0.27 mm and about 0.36 mm, between about 0.29 mm and about 0.33 mm. In some examples, the angled struts 704b can have a width 722 of about 0.3 mm. In some examples, the width 724 of the angled struts 704c can be between about 0.31 and about 0.41 mm, between about 0.33 mm and about 0.39 mm, between about 0.35 mm and about 0.37 mm. In some examples, the width 724 of the angled struts 704c can be about 0.36 mm.

The outflow angled struts 704a can have a width 726 and the inflow angled struts 704d can have a width 728. In some examples, such as the example illustrated in FIG. 14, the width 726 and the width 728 can be equal. For example, the widths 726 and 728 can be between about 0.34 mm and about 0.45 mm, between about 0.37 mm and about 0.43 mm, between about 0.39 mm and about 0.41 mm. In some examples, each width 726 and 728 can be about 0.4 mm.

In some examples, a frame can be comprised of multiple rows of struts each of which have a different width. For example, FIG. 15 illustrates another example of frame 700, referred to as frame 700′ having struts 704′. Frame 700′ is the same as frame 700 except where indicated otherwise. Frame 700′ includes first, second, third, and fourth rows of struts 704a′-704d′, each of which has a different width 722′, 724′, 726′, and 728′ respectively. In some examples, the angled struts 704b′ can have a width 722′ of about 0.32 mm, the angled struts 704c′ can have a width 724′ of about 0.38 mm, the outflow angled struts 704a′ can have a width 726′ of about 0.36 mm, and the inflow angled struts 704d′ can have a width 728′ of about 0.4 mm.

In some examples, each row of angled struts 704a-704d (or 704a′-704b′) can have struts having a different length relative to the length of the struts in the other rows of angled struts. The strut lengths can be selected to provide a desired profile for the expanded valve, such as a V-shaped, or A-shaped frame configuration. For example, shorter struts will provide greater resistance during expansion. For example, frames 700 and 700′ can be configured (e.g., the lengths of the intermediate angled struts 704a-704d and 704a′-704d′ can be selected) to result in a V-shaped frame when in the expanded configuration. In some examples, the outflow row of struts 704a and 704a′ can have a length between about 2 mm and about 5 mm and the inflow row of struts 704d and 704d′ can likewise have a length between about 2 mm and about 5 mm. In some examples, the intermediate angled rows of struts, e.g., the second and third rows 704b/704b′ and 704c/704c′, can have a length between about 3 mm and about 8 mm.

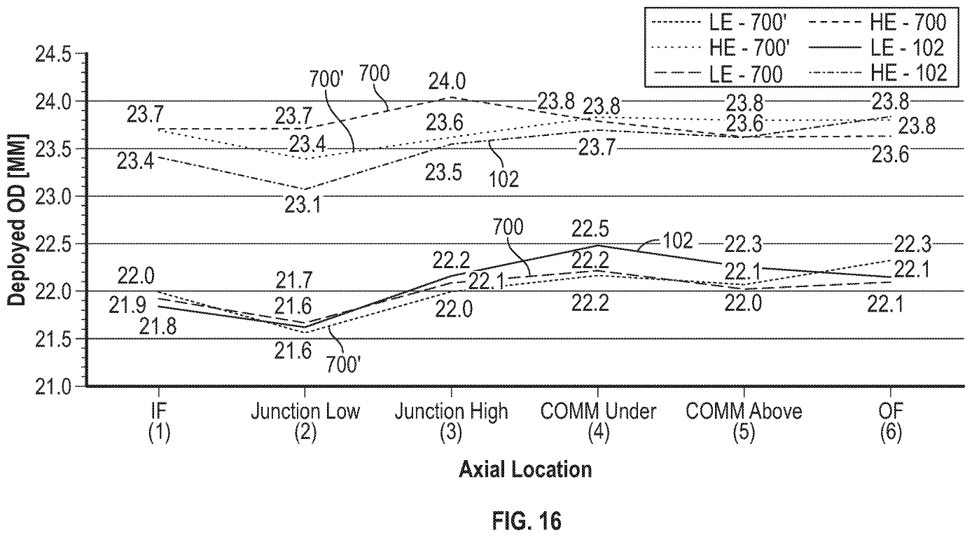

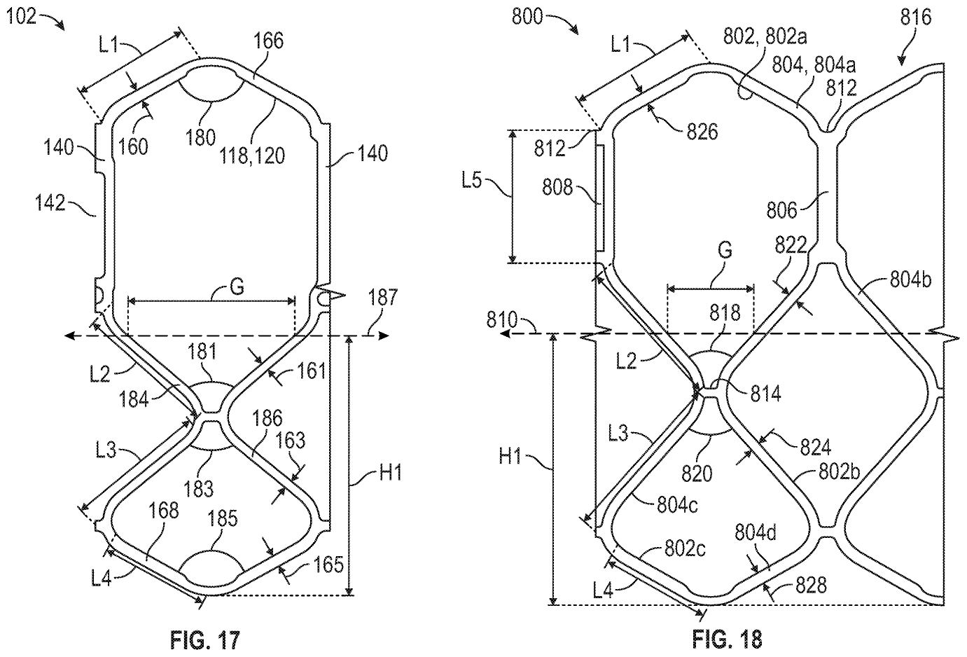

FIG. 16 illustrates a graph of the expanded frame diameter of frames 700 (FIGS. 14), 700′ (FIG. 15), and 102 (FIG. 17) at different axial levels of the frame (indicated on frame 700 in FIG. 14): (1) at the inflow end, (2) at the lower strut junction, (3) at the higher strut junction, (4) under the commissure, (5) above the commissure, and (6) at the outflow end. The three lines at the upper half of the graph (indicated with the legend HE for “higher end”) illustrate the higher end of the expansion diameters for each frame 700/700′/102. The three lines at the bottom half of the graph (indicated with the legend “LE” for “lower end”) illustrate the lower end of the expansion diameters for each frame 700/700′/102.

As shown, the frames 700 and 700′ have an outflow diameter (6) that is greater than the inflow diameter (1), resulting in a V-shaped configuration that can advantageously improve pressure gradients across the prosthetic valve after implantation. For example, in some cases, frame 700 can have an inflow diameter of about 21.9 mm and an outflow diameter of about 22.1 mm, and frame 700′ can have an inflow diameter of about 22.0 mm and an outflow diameter of about 22.3 mm. In some examples, the V-shaped configuration can be the result of the inflow end struts having a greater width than the remaining struts. In other examples, such as frames 700 and 700′, the V-shaped configuration can be the result of varying the width along each row of struts 704a-704d/704a′-704d′.

FIG. 17 illustrates a portion of frame 102 described previously. As shown in FIG. 17, the frame 102 can comprise a first row of angled struts 166 at the outflow end (also referred to as outflow struts), a row of axial struts 140, some of which contain commissure windows 142, a second row of angled struts 184, a third row of angled struts 186, and a fourth row of angled struts 168 at the inflow end (also referred to as inflow struts). As mentioned previously, the outflow angled struts can form a first angle 180 between two adjacent struts. A second angle 181 can be formed between adjacent struts 184 of the second row of angled struts, a third angle 183 can be formed between adjacent struts 186 of the third row of angled struts, and a fourth angle 185 can be formed between adjacent inflow struts 168. In the example illustrated in FIG. 17, the first angle 180 can be about 120 degrees, the second angle 181 can be about 100 degrees, the third angle 183 can be about 100 degrees, and the fourth angle 185 can be about 120 degrees.

Each strut 166 of the first row of angled struts 166 can have a first length L1, each strut 184 of the second row of angled struts can have a second length L2, each strut 186 of the third row of angled struts can have a third length L3, and each strut 168 of the fourth row of angled struts can have a fourth length L4. In some examples, the first and fourth lengths L1, L4 can be equal, in other examples, the lengths L1 and L4 can be different from one another. The second and third lengths L2, L3 can also be equal in some examples, and in other examples, they can be different from one another.

Each strut 166 of the first row of struts can have a first width 160, each strut 184 of the second row of struts can have a second width 161, each strut 186 of the third row of struts can have a third width 163, and each strut 168 of the fourth row of struts can have a fourth width 165. In some examples, the first and fourth widths 160, 165 are equal to one another and the second and third widths 161, 163 are equal to one another. For example, in FIG. 17, the widths 160 and 165 can each be about 0.4 mm, and the widths 161, 163 can each be about 0.3 mm.

In other examples, such as frame 700′ illustrated in FIG. 15, the first and fourth widths 726′ and 728′ can be different from one another. For example, width 726′ can be about 0.36 mm and width 728′ can be about 0.4 mm. In some examples, the second and third widths can also be different from one another, such as shown in frames 700 and 700′. For example, frame 700 comprises second struts 704b having a width 722 of about 0.3 mm and third struts 704c having a width 724 of about 0.36 mm, and frame 700′ comprises second struts 704b′ having a width 722′ of about 0.32 mm and third struts 704c′ having a width 724′ of about 0.38 mm.

FIG. 18 illustrates another example of a frame 800 that can be used as part of a prosthetic heart valve, such as prosthetic heart valve 100 described previously. The frame 800 can be similar to frame 102, except where indicated otherwise. For example, frame 800 can comprise three circumferentially extending rows of cells 802 (including an outflow end row of cells 802a, a middle row of cells 802b, and an inflow end row of cells 802c) formed from four rows of angled struts 804, namely angled struts 804a-804d and a row of axially extending struts 806 selected ones of which include a commissure window 808. The prosthetic valve can comprise a valvular structure such as valvular structure 104 described previously, which can be secured to and supported within the frame 800.

In some examples, the prosthetic valve can comprise an inner skirt or sealing member disposed on the radially inner surface of the frame. In some examples, the inner skirt can comprise an outflow edge having a zig-zag shape such that the outflow edge extends along the struts 804b of the second row of angled struts.

In some examples, the prosthetic valve can further comprise an outer skirt or sealing member disposed on an outer surface of the frame. In some examples, the sealing member can have an outflow edge that terminates just below the vertically extending struts of the frame, such as illustrated by dashed line 187 in FIG. 17. In some examples, the sealing member can have a height H1 of about 9 mm as measured from the inflow end of the frame.

Referring to FIG. 18, in some examples, the frame 800 of the prosthetic valve can be configured such that an outflow edge of the sealing member (indicated by dashed line 810) terminates approximately halfway along the second row of struts 804b. This advantageously prevents the outflow end portion of the sealing member from protruding through the outflow row of cells 802a, thereby preventing or mitigating contact between the sealing member and the valvular structure, thereby reducing the risk of leaflet abrasion.

As shown in FIG. 18, the frame 800 has axially extending struts 806 that are relatively shorter when compared to frame 102 such that the commissure window 808 has been shifted toward the outflow end of the frame when compared to frame 102. For example, axially extending struts 806 can have a length L5 of between about 4.5 mm and about 9 mm. The commissure window 808 can be disposed within the axially extending strut 806 such that it is between about 0.3 mm and about 3 mm from the outflow edge 812 of the axially extending strut 806, resulting in a commissure window 808 that is shifted toward the outflow end of the frame about 1.14 mm relative to the commissure window 142 in frame 102. Such a configuration can result in the intermediate angled struts 804b and 804c being elongated relative to the intermediate angled struts 184, 186 shown in frame 102.

In some examples, angled struts 804b can have a length L2 of between about 3 mm and about 8 mm. In some examples, angled struts 804c can have a length L3 between about 3 mm and about 8 mm. Such a configuration can also result in the inflow apices 814 of the first row of cells 802a being positioned closer to the outflow end 816 of the frame 800 when compared to the inflow apices of the first row of cells of, for example, frame 102 shown in FIG. 17. In some examples, the angle 818 between adjacent intermediate angled struts 804b can be between about 80 degrees and 125 degrees, and the angle 820 between adjacent intermediate angled struts 804c can be between about 60 degrees and about 100 degrees. For example, FIG. 18 illustrates an embodiment of frame 800 wherein the angle 818 is about 84 degrees and the angle 820 is about 84 degrees.

Each strut 804a of the first row of struts can have a first width 826, each strut 804b of the second row of struts can have a second width 822, each strut 804c of the third row of struts can have a third width 824, and each strut 804d of the fourth row of struts can have a fourth width 828. In some examples, the first and fourth widths 826, 828 are equal to one another and the second and third widths 822, 824 are equal to one another. For example, in FIG. 18, the widths 826, 828 can each be about 0.4 mm, and the widths 822, 824 can each be about 0.3 mm.

As shown in FIGS. 17-18, the distance between adjacent intermediate angled struts 184 or 804b at the location where the outflow edge 187 or 810 of the sealing member crosses the cell 120 or 802a can be referred to herein as the “sealing member gap” G. The examples shown in FIGS. 14-15 and 19-20 illustrate exemplary configurations of frames that advantageously minimize the sealing member gap G, thereby preventing the outflow end portion of the sealing member from protruding through the outflow row of cells.

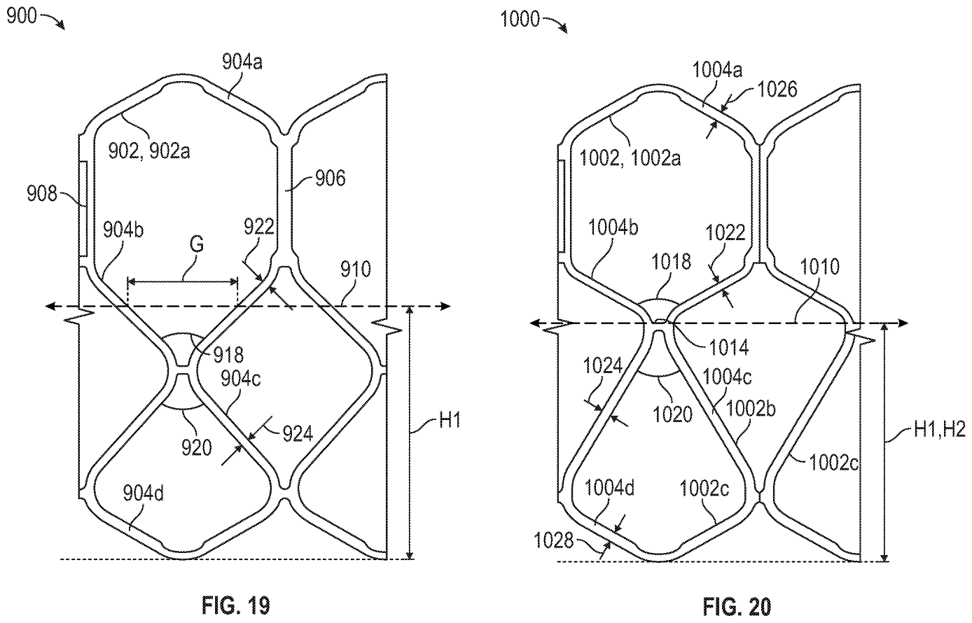

Frames 900-1000 are similar to frames 700/800, having a plurality of cells 902/1002 (including an outflow row of cells 902a/1002a) formed from four rows of angled struts 904/1004, namely angled struts 904a-904d/1004a-1004d and a row of axially extending struts 906/1006 selected ones of which include a commissure window 908/1008. FIGS. 19-20 each include a dashed line 910/1010 representing the outflow edge of the sealing member having a height H1.

The frames 700, 700′, 800, 900, and 1000 can be configured such that the sealing member gap G is between about 2.5 mm and about 3.4 mm. These configurations advantageously prevent or mitigate protrusion of the sealing member through the first row of cells 702a/802a/902a/1002a without reducing the height of the sealing member, thereby allowing the sealing member to provide adequate PVL sealing. In some examples, the height H1 of the sealing member is about 9 mm.

Referring again to FIGS. 14-15, which illustrate frames 700 and 700′, in some examples the frame 700/700′ can comprise an angle 719 between adjacent struts 704b/704b′ of the second row of struts and an angle 721 between adjacent struts 704c/704c′ of the third row of struts. In some examples, the angle 719 can be about 100 degrees, and the angle 721 can be about 79 degrees. Such a configuration results in a sealing member gap G of about 2.8 mm.

As mentioned previously, FIG. 18 illustrates frame 800 wherein the angle 818 between adjacent intermediate angled struts 804b is about 84 degrees and the angle 820 between adjacent intermediate angled struts 804c is also about 84 degrees. As mentioned previously, the length L2 of the struts 804b can be between about 3 mm and about 8 mm, and the width 822 is about 0.34 mm. The length L3 of the struts 804c can be between about 3 mm and about 8 mm, and the width 824 is about 0.34 mm. Such a configuration results in a sealing member gap G of about 2.9 mm.

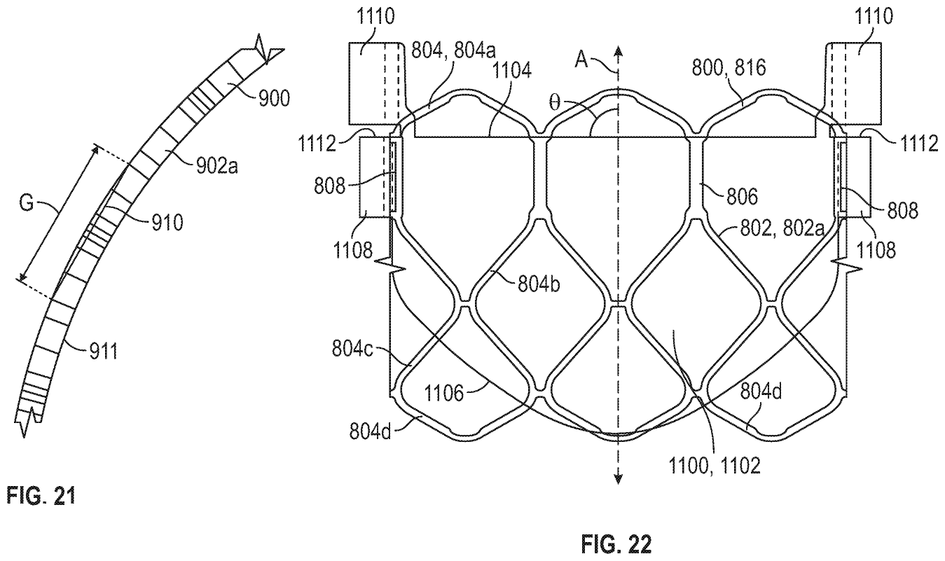

FIG. 19 illustrates another exemplary frame 900 that is similar to frames 102/800 except that the commissure window 908 is shifted toward the outflow end of the frame 900 by about 0.6 mm and the axially-extending struts 906 are shortened, resulting in an angle 918 of about 93 degrees and an angle 920 of about 85 degrees. This configuration results in a sealing member gap G of about 3.35 mm. FIG. 21 illustrates a cross-section of cell 902a of frame 900, showing the outflow edge 910 of the sealing member at the sealing member gap G. As shown in FIG. 21, the sealing member gap G is narrow enough to prevent the sealing member 910 from protruding radially inwardly past the radially inner surface 911 of the frame 900. This mitigates contact between the valvular structure and the sealing member, thereby increasing the longevity of the leaflets.

FIG. 20 illustrates another exemplary frame 1000 wherein the intermediate angled struts 1004b are shortened relative to the other frames, such as frames 102, 800 and 900, and the intermediate angled struts 1004c are elongated resulting in a frame 1000 having a shortened outflow row of cells 1002a and an elongated inflow row of cells 1002c. For example, the intermediate angled struts 1004b can have a length L2 of between about 3 mm and about 8 mm and the angled struts 1004c can have a length L3 of between about 3 mm and about 8 mm. In such examples, each inflow cell 1002c can have a height H2 of about 9 mm, commensurate with the height H1 of the sealing member.

Each strut 1004a of the first row of struts can have a first width 1026, each strut 1004b of the second row of struts can have a second width 1022, each strut 1004c of the third row of struts can have a third width 1024, and each strut 1004d of the fourth row of struts can have a fourth width 1028. In some examples, the first and fourth widths 1026, 1028 can be equal to one another. For example, the first and fourth widths 1026, 1028 can be about 0.4 mm. In other examples, the first and fourth widths 1026, 1028 can be different from one another. In some examples, the second and third widths 1022, 1024 can be equal to one another. In other examples, such as the example shown in FIG. 20, the second and third widths 1022, 1024 can be different from one another. For example, in FIG. 20, the second width 1022 is about 0.28 mm and the third width 1024 is about 0.38 mm.

The angle 1018 between adjacent struts 1004b can be about 123 degrees, and the angle 1020 between adjacent struts 1004c can be about 63 degrees. Such a configuration advantageously results in no sealing member gap G, since the outflow edge 1010 of the sealing member is positioned substantially level with the inflow apex 1014 of each cell 1002a of the outflow row of cells. Further, the larger angles between the adjacent intermediate angled struts 1004a, 1004b, and 1004d can provide additional stabilization to the frame, mitigating the risk of regional under-deployment. “Regional under-deployment” as used herein refers to a configuration wherein one or more columns of cells is under-deployed relative to the neighboring columns (i.e., the angles between adjacent struts have not reached a desired size). Regional under-deployment can result in undesirable patient outcomes because the leaflets can contact the under-deployed portions of the frame, causing wear, and also because the under-deployment results in reduced leaflet functionality as each leaflet will not cover the necessary 120 degrees of the circumference of the valve. The frames disclosed herein advantageously mitigate or prevent these issues.

As mentioned previously, in some examples, the angled struts of each row of struts can have a different length and/or width from the angled struts of each other row of struts. In some examples, the angles between each pair of adjacent struts in the intermediate rows of struts can differ from row to row. Such a configuration is illustrated, for example, in frame 700′ shown in FIG. 15. As discussed previously, frame 700′ has a first (or outflow) row of struts 704a′ each having a width 726′ of about 0.36 mm, a second row of struts 704b′ each having a width 722′ of about 0.32 mm, a third row of struts 704c′ each having a width 724′ of about 0.38 mm, and a fourth (or inflow) row of struts 704d′ each having a width 728′ of about 0.4 mm. The angle 719 between adjacent struts 704b′ of the second row of struts is different from the angle 721 between adjacent struts 704c′ of the third row of struts. For example, angle 719 can be about 100 degrees, and angle 721 can be about 79 degrees. Such a configuration can influence the radial resistance of the frame. For example, wider angles between adjacent struts can increase the stiffness of the frame, which can advantageously withstand radial resistive and crushing forces and increase the longevity of the valvular structure, particularly in prosthetic valves that are expanded to relatively larger diameters (e.g., from about 29 mm to about 31 mm).

In some examples, the frame can be configured to maintain a cylindrical configuration during expansion by having the widths of the struts in each row of struts be relatively greater or lesser depending on the selected angle between adjacent struts. For example, angled struts that form a row that defines relatively wider angles between the struts can have a lesser width, and angled struts that form a row that defines relatively narrower angles between the struts can have a greater width.

For example, FIG. 19, illustrates a frame 900 wherein the width of the intermediate angled struts 904b, 904c is dictated by the angles between adjacent struts 904b, 904c in the second and third rows, respectively. The struts 904b in the second row, which have an angle 918 between them of about 93 degrees, can have a width 922 that is less than the width 924 of struts 904c in the third row, which have an which have an angle 920 between them of about 85 degrees.

Though the exemplary frames 102, 700-1000 illustrated herein are nine cell frames, it should be understood that the principles discussed (e.g., struts having a different angle between them in some or all rows, struts having a different length in some or all rows, struts having a different width in some or all rows, etc.) can also apply to twelve cell frames, or frames of other sizes.

In some instances, after a prosthetic heart valve has been implanted it may become necessary to access the coronary artery. Thus, it may be necessary to preserve coronary access through the frame of a prosthetic heart valve, such as through outflow row of cells between the outflow end of the frame and the outflow end of the valvular structure. This access can be affected by the positioning of the commissure window, which can affect the axial positioning of the valvular structure within the frame. FIG. 22 illustrates an exemplary leaflet 1100 configured to be used with frame 800 (FIG. 18) to preserve coronary access. In other examples, leaflet 1100 can be configured to be used with frames 700, 700′, 900, 1000. FIG. 22 illustrates the leaflet in a flattened configuration disposed behind a portion of the frame 800 (also shown in a flattened configuration).

Referring to FIG. 22, the leaflet 1100 can comprise a main body 1102, an outflow edge portion 1104 (also referred to as a coaptation edge), a lower edge portion 1106 (also referred as the cusp edge or scallop edge) terminating at laterally projecting tabs 1108 (also referred to as commissure tabs). In some examples, such as the illustrated example, the tabs 1108 can be formed integrally with the main body 1102 of the leaflet 1100. In some examples, the leaflet 1100 can further comprise upper tabs 1110, which can, in some examples, be folded together with tabs 1108 to form part of the commissure. Tabs 1108 of adjacent leaflets can be coupled together to form a commissure, which can be coupled to the frame 800 at a commissure window 808 (partially shown).

As shown in FIG. 22, the outflow or coaptation edge 1104 of the leaflet 1100 can be disposed at an angle θ relative to a longitudinal axis A of the leaflet 1100. In some examples, the angle θ can be a 90 degree angle, resulting in the outflow edge 1104 of the leaflet 1100 being aligned (e.g., colinear) with the outflow edges 1112 of the leaflet tabs 1108. This configuration can advantageously provide sufficient space between the outflow end 816 of the frame 800 and the outflow edge 1104 of the leaflet 1100 to allow for coronary access. For example, the gap between the outflow edge 1104 of the leaflet and the outflow end of the frame 800 can be large enough to allow a coronary catheter to pass through (e.g., a 6 Fr coronary catheter), thereby allowing access to the coronary ostia.

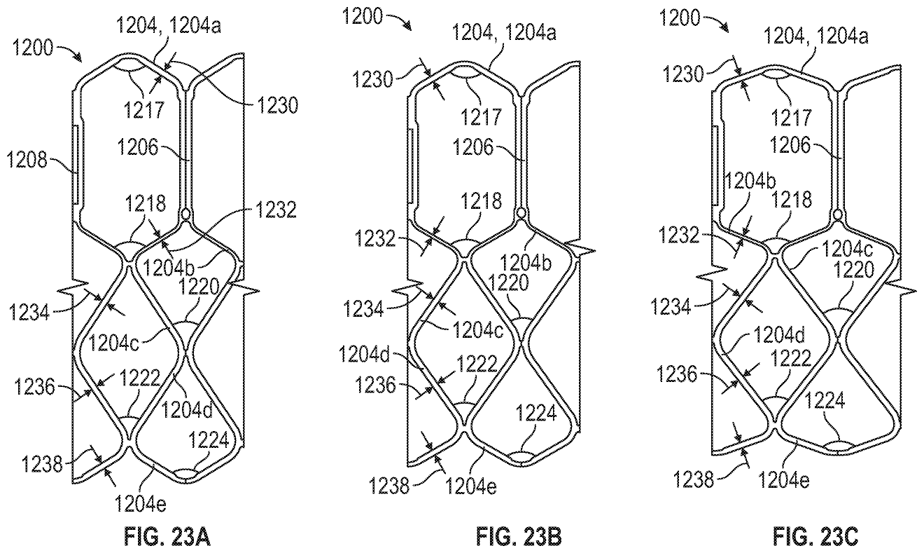

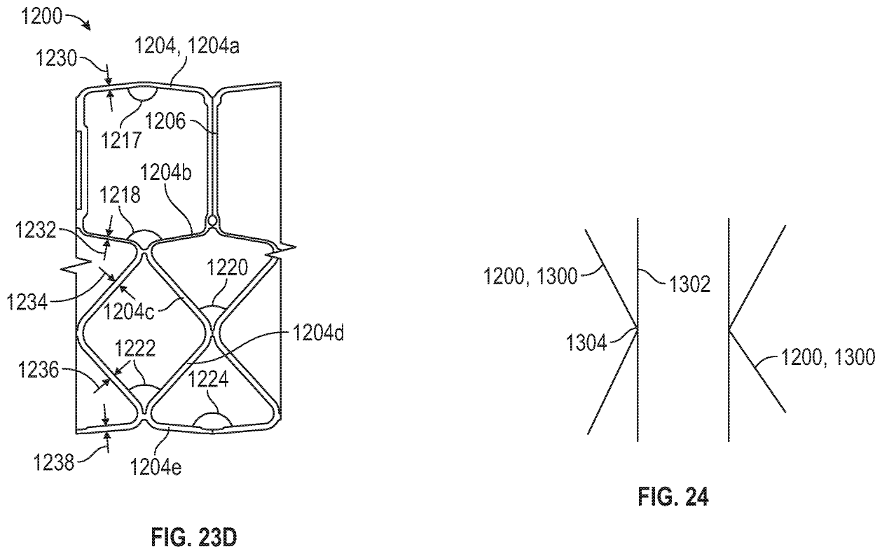

FIGS. 23A-23D illustrate an exemplary frame 1200 having a plurality of cells 1202 formed by five rows of angled struts 1204 (rows 1204a-1204e) and a row of axial struts 1206, some of which include commissure windows 1208. The frame 1200 is shown in various radially expanded working states, such as in a 26.2 mm working diameter in FIG. 23A, a 27 mm working diameter in FIG. 23B, a 29 mm working diameter in FIG. 23C, and a 31 mm working diameter in FIG. 23D.

In some examples, as the frame 1200 is expanded to a larger diameter the angles between adjacent struts along an intermediate row of struts, such as the second row of struts 1204b, can be enlarged to allow the intermediate row 1204b to assume a relatively flattened configuration (e.g., nearly perpendicular to a central axis of the frame 1200) when the frame 1200 is over-expanded, such as in a valve-in-valve (ViV) procedure.

In some examples, the first row of struts 1204a can have a width 1230 of about 0.36 mm, the second row 1204b can have a width 1232 of about 0.24 mm, the third row 1204c can have a width 1234 of about 0.36 mm, the fourth row 1204d can have a width 1236 of about 0.36 mm, and the fifth row 1204e can have a width 1238 of about 0.35 mm.

In some examples, the first row of struts 1204a can have a length of between about 2 mm and about 5 mm, the second row 1204b can have a length of between about 3 mm and about 8 mm, the third row 1204c can have a length of between about 3 mm and about 8 mm, the fourth row 1204d can have a length of between about 3 mm and about 8 mm, and the fifth row 1204e can have a length of about 2 mm and about 5 mm.

FIG. 23A illustrates a frame 1200 expanded to a diameter of about 26.2 mm, resulting in a first angle 1217 of about 115 degrees, a second angle 1218 of about 116 degrees, a third angle 1220 of about 67 degrees, a fourth angle 1222 of about 67 degrees, and a fifth angle 1224 of about 116 degrees. FIG. 23B illustrates the frame 1200 expanded to a diameter of about 27 mm, which results in a first angle 1217 of about 123 degrees, a second angle 1218 of about 123 degrees, a third angle 1220 of about 70 degrees, a fourth angle 1222 of about 70 degrees, and a fifth angle 1224 of about 123 degrees. FIG. 23C illustrates the frame 1200 expanded to a diameter of about 29 mm resulting in a first angle 1217 of about 142 degrees, a second angle 1218 of about 140 degrees, a third angle 1220 of about 78 degrees, a fourth angle 1222 of about 78 degrees, and a fifth angle 1224 of about 142 degrees. FIG. 23D illustrates the frame 200 expanded to a diameter of about 31 mm resulting in a first angle 1217 of about 169 degrees, a second angle 1218 of about 163 degrees, a third angle 1220 of about 86 degrees, a fourth angle 1222 of about 87 degrees, and a fifth angle 1224 of about 170 degrees.

As shown in FIG. 23D, when the frame is expanded to a relatively larger diameter, such as about 31 mm, the second row of struts 1204b are in a flattened configuration wherein they extend nearly perpendicularly relative to a central axis of the frame 1200. Accordingly, the second row of struts 1204b serves as a limiting factor that prevents expansion of the frame past a selected diameter (constrained by the length of the struts 1204b) at the axial height of the second row of struts 1204b.

The second row of struts 1204b can therefore be referred to as an “anchoring belt” because it can improve anchoring between a guest valve and a host valve in a valve-in-valve procedure. For example, FIG. 24 illustrates an example wherein a prosthetic valve comprising frame 1200 is used as a host valve 1300 for a guest valve 1302. The host valve 1300 can assume an hourglass configuration wherein the narrower neck portion 1304 of the hourglass shape is caused by the second row of struts 1204b, which prevent expansion of the host frame 1200 past a selected diameter at that axial location.

In some examples, such as illustrated in FIGS. 23A-23D, the angles 1217 and 1224 between adjacent struts at the outflow row of struts 1204a and the inflow row of struts 1204e can also be relatively larger (e.g., when compared to previously disclosed examples). Such a configuration can result in three rows of struts (the outflow row of struts 1204a, the second row of struts 1204b, and the inflow row of struts 1204e) each serving as anchoring belts for the frame 1200. In other examples, such as shown in FIG. 24, only the second row of struts 1204b serves as an anchoring belt, allowing the inflow and outflow rows of struts 1204a, 1204e to expand to a diameter greater than the diameter of the second row of struts 1204b, thereby resulting in an hourglass shape for the expanded frame 1200.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve, comprising: a radially expandable and compressible annular frame comprising a plurality of interconnected angled struts defining a plurality of circumferentially extending rows of cells arranged between an outflow end and an inflow end of the frame; wherein the plurality of interconnected angled struts is arranged to form a plurality of circumferentially extending rows of struts, including an outflow row of struts at the outflow end of the frame, one or more intermediate rows of struts, and an inflow row of struts at the inflow end of the frame, the one or more intermediate rows of struts disposed between the outflow row of struts and the inflow row of struts; wherein each strut of the outflow row of struts has a width greater than a width of each strut of the one or more intermediate rows of struts; wherein the plurality of circumferentially extending rows of cells comprises a first row of first cells disposed at the outflow end and at least partially defined by the first and second rows of struts, and a second row of second cells disposed adjacent to the first row of first cells and at least partially defined by the second and third rows of struts, wherein each first cell has a first axial length that is longer than a second axial length of each second cell.

Example 2. The prosthetic heart valve of any example herein, particularly example 1, wherein each strut of the inflow row of struts has a width greater than the width of each strut of the one or more intermediate rows of struts and less than the width of each strut of the outflow row of struts.

Example 3. The prosthetic heart valve of any example herein, particularly any one of examples 1-2, wherein the width of each strut of the outflow row of struts is between 0.32 mm and 0.44 mm.

Example 4. The prosthetic heart valve of any example herein, particularly any one of examples 1-3, wherein the width of each strut of the outflow row of struts is 0.4 mm.

Example 5. The prosthetic heart valve of any example herein, particularly any one of examples 1-4, wherein the width of each strut of the one or more intermediate rows of struts is between 0.22 mm and 0.34 mm.

Example 6. The prosthetic heart valve of any example herein, particularly any one of examples 1-5, wherein the width of each strut of the one or more intermediate rows of struts is 0.28 mm.

Example 7. The prosthetic heart valve of any example herein, particularly any one of examples 1-6, wherein the width of each strut of the inflow row of struts is between 0.3 mm and 0.42 mm.

Example 8. The prosthetic heart valve of any example herein, particularly any one of examples 1-7, wherein the width of each strut of the inflow row of struts is 0.37 mm.

Example 9. The prosthetic heart valve of any example herein, particularly any one of examples 1-8, wherein the outflow row of struts is a first row of struts, the one or more intermediate rows of struts comprises second, third, and fourth rows of struts, and the inflow row of struts is a fifth row of struts.

Example 10. The prosthetic heart valve of any example herein, particularly any one of examples 1-9, wherein the plurality of circumferentially extending rows of cells comprises four rows of cells.

Example 11. The prosthetic heart valve of any example herein, particularly any one of examples 1-10, further comprising a valvular structure disposed within and coupled to the annular frame.

Example 12. The prosthetic heart valve of any example herein, particularly any one of examples 1-11, wherein the outflow row of struts form pairs of angled struts, wherein the struts of each pair of angled struts are connected together at their adjacent ends by an outflow apex region, and wherein the outflow apex region curves between the pair of angled struts and has a narrowed width relative to each strut of the pair of angled struts.

Example 13. The prosthetic heart valve of any example herein, particularly any one of examples 1-12, wherein the frame further comprises a plurality of axially extending struts that extend between the outflow row of struts and a first row of the one or more intermediate rows of struts and define axial sides of a first row of first cells.

Example 14. The prosthetic heart valve of any example herein, particularly example 13, wherein a portion of the plurality of axially extending struts are axially extending window struts that define commissure windows, further comprising a plurality of leaflets secured together at their adjacent sides to form commissures, and wherein the commissures are secured to the commissure windows of the frame.

Example 15. A prosthetic heart valve, comprising: a radially expandable and compressible annular frame comprising a plurality of interconnected angled struts defining a plurality of circumferentially extending rows of cells arranged between a first end and a second end of the frame; and a plurality of leaflets disposed within the frame and secured together at their adjacent sides to form commissures; wherein the plurality of interconnected angled struts is arranged to form a plurality of circumferentially extending rows of angled struts, including a first row of angled struts at the first end of the frame, one or more intermediate rows of angled struts, and a second row of angled struts at the second end of the frame, the one or more intermediate rows of angled struts disposed between the first row of angled struts and the second row of angled struts; wherein each strut of the first row of angled struts has a width greater than a width of each strut of the one or more intermediate rows of angled struts and each strut of the second row of angled struts; wherein the frame further comprises a plurality of axially extending struts that extend between the first row of angled struts at the first end of the frame and a first row of the one or more intermediate rows of angled struts and define axial sides of a first row of first cells; wherein a portion of the plurality of axially extending struts are axially extending window struts that define commissure windows; and wherein the commissures are secured to the commissure windows of the frame.

Example 16. The prosthetic heart valve of any example herein, particularly example 15, wherein the width of each strut of the second row of angled struts is greater than the width of each strut of the one or more intermediate rows of angled struts.

Example 17. The prosthetic heart valve of any example herein, particularly any one of examples 15-16, wherein the width of each strut of the second row of angled struts is less than the width of each strut of the outflow row of struts.

Example 18. The prosthetic heart valve of any example herein, particularly any one of examples 15-17, wherein the width of each strut of the first row of angled struts is 0.4 mm.

Example 19. The prosthetic heart valve of any example herein, particularly any one of examples 15-18, wherein the width of each strut of the one or more intermediate rows of angled struts is 0.28 mm.

Example 20. The prosthetic heart valve of any example herein, particularly any one of examples 15-19, wherein the width of each strut of the second row of angled struts is 0.37 mm.

Example 21. A prosthetic heart valve, comprising: a radially expandable and compressible annular frame comprising a plurality of interconnected angled struts defining four circumferentially extending rows of cells arranged between a first end and a second end of the frame; wherein the plurality of interconnected angled struts is arranged to form five circumferentially extending rows of angled struts, including a first row of angled struts at the first end of the frame, second, third, and fourth rows of angled struts, and a fifth row of angled struts at the second end of the frame, the second, third, and fourth rows of angled struts disposed between the first row of angled struts and the fifth row of angled struts; wherein the first row of angled struts is comprised of pairs of angled struts, each pair of angled struts in the first row being connected together at their adjacent ends by an apex region, and wherein the apex region curves between the pair of angled struts and has a narrowed width relative to a respective width of each strut of the pair of angled struts; wherein the width of each strut of the first row of angled struts is greater than a width of each strut of the one or more intermediate rows of angled struts and each strut of the second row of angled struts.

Example 22. The prosthetic heart valve of any example herein, particularly example 21, wherein the four circumferentially extending rows of cells include a first row of first cells at the first end of the frame, a second row of second cells, a third row of third cells, and a fourth row of fourth cells at the second end of the frame, wherein each first cell has a first axial length that is longer than a second axial length of each second cell.

Example 23. A prosthetic heart valve, comprising: a radially expandable and compressible annular frame comprising a plurality of interconnected angled struts defining a plurality of circumferentially extending rows of cells arranged between an outflow end and an inflow end of the frame; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein each strut of the first row of struts has a first width, each strut of the second of struts has a second width, each strut of the third row of struts has a third width, and each strut of the fourth row of struts has a fourth width; and wherein the second and third widths are different from one another.

Example 24. The prosthetic heart valve any example herein, particularly example 23, wherein the first and fourth widths are different from one another.

Example 25. The prosthetic heart valve of any example herein, particularly example 23, wherein the first and fourth widths are equal.

Example 26. The prosthetic heart valve of any example herein, particularly any one of examples 23-24, wherein the first, second, third, and fourth widths are each different from one another.

Example 27. The prosthetic heart valve of any example herein, particularly any one of examples 23-26, wherein the second width is between about 0.25 mm and about 0.35 mm.

Example 28. The prosthetic heart valve of any example herein, particularly any one of examples 23-27, wherein the second width is about 0.3 mm.

Example 29. The prosthetic heart valve of any example herein, particularly any one of examples 23-28, wherein the third width is between about 0.31 mm and about 0.41 mm.

Example 30. The prosthetic heart valve of any example herein, particularly any one of examples 23-29, wherein the third width is about 0.36 mm.

Example 31. The prosthetic heart valve of any example herein, particularly any one of examples 23-30, wherein the first width is about 0.4 mm.

Example 32. The prosthetic heart valve of any example herein, particularly any one of examples 23-31, wherein the first width is about 0.36 mm.

Example 33. The prosthetic heart valve of any example herein, particularly any one of examples 23-32, wherein the fourth width is about 0.4 mm.

Example 34. The prosthetic heart valve of any example herein, particularly any one of examples 23-33, wherein when the frame is in a radially expanded configuration the outflow end portion of the frame has a first diameter and the inflow end portion of the frame has a second diameter, and wherein the first diameter is larger than the second diameter.

Example 35. The prosthetic heart valve of any example herein, particularly example 34, wherein the first diameter is about 22.1 mm and the second diameter is about 21.9 mm.

Example 36. The prosthetic heart valve of any example herein, particularly example 34, wherein the first diameter is about 22.3 mm and the second diameter is about 22.0 mm.

Example 37. The prosthetic heart valve of any example herein, particularly any one of examples 23-36, wherein each strut of the first row of struts has a first length, each strut of the second of struts has a second length, each strut of the third row of struts has a third length, and each strut of the fourth row of struts has a fourth length; and wherein the first length, second length, third length, and fourth length are each different from one another.

Example 38. The prosthetic heart valve of any example herein, particularly any one of examples 23-37, wherein each strut of the first row of struts has a first width, each strut of the second of struts has a second width, each strut of the third row of struts has a third width, and each strut of the fourth row of struts has a fourth width; and wherein the first width, second width, third width, and fourth width are each different from one another.

Example 39. The prosthetic heart valve of any example herein, particularly example 38, wherein the second width is between about 0.27 mm and about 0.35 mm.

Example 40. The prosthetic heart valve of any example herein, particularly any one of examples 38-39, wherein the third width is between about 0.3 mm and 0.39 mm.

Example 41. The prosthetic heart valve of any example herein, particularly any one of examples 23-40, further comprising a sealing member disposed on a radially outward facing surface of the frame.

Example 42. The prosthetic heart valve of any example herein, particularly example 41, wherein the sealing member has a height of about 9 mm.

Example 43. The prosthetic heart valve of any example herein, particularly any one of examples 41-42, wherein the plurality of circumferentially extending rows of cells comprises an outflow row of cells disposed at the outflow end of the frame, wherein the sealing member crosses the outflow row of cells at a selected height, and wherein the distance between two adjacent angled struts of the second row of angled struts at the selected height is the sealing member gap.

Example 44. The prosthetic heart valve of any example herein, particularly example 43, wherein the sealing member gap is between about 2.5 mm and about 3.4 mm.

Example 45. The prosthetic heart valve of any example herein, particularly any one of examples 23-44, further comprising a plurality of axially-extending struts that extend from the first row of angled struts to the second row of angled struts.

Example 46. The prosthetic heart valve of any example herein, particularly any one of examples 23-45, wherein each pair of adjacent struts in the first row of struts has a first angle between them, each pair of adjacent angled struts in the second row of struts has a second angle between, each pair of adjacent struts in the third row of struts has a third angle between them, and each pair of adjacent struts in the fourth row of struts has a fourth angle between them.

Example 47. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; and a sealing member disposed on a radially outwardly facing surface of the frame; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein each strut of the first row of struts has a first length, each strut of the second row of struts has a second length, each strut of the third row of struts has a third length, and each strut of the fourth row of struts has a fourth length; and wherein a height of the sealing member is equal to a height of the inflow row of cells.

Example 48. The prosthetic heart valve of any example herein, particularly example 47, wherein a first angle between adjacent struts in the second row of struts is about 123 degrees and wherein a second angle between adjacent struts in the third row of struts is about 63 degrees.

Example 49. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein each strut of the first row of struts has a first width, each strut of the second row of struts has a second width, each strut of the third row of struts has a third width, and each strut of the fourth row of struts has a fourth width; wherein each pair of adjacent struts of the first row of struts has a first angle between them, each pair of adjacent struts of the second row of struts has a second angle between them, each pair of adjacent struts of the third row of struts has a third angle between them, and each pair of adjacent struts of the fourth row of struts has a fourth angle between them; and wherein the second and third angles are different from one another and the second and third widths are different from one another.

Example 50. The prosthetic heart valve of any example herein, particularly example 49, wherein the second and third widths are based on the second and third angles.

Example 51. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein each strut of the first row of struts has a first length, each strut of the second row of struts has a second length, each strut of the third row of struts has a third length, and each strut of the fourth row of struts has a fourth length; wherein each pair of adjacent struts of the first row of struts has a first angle between them, each pair of adjacent struts of the second row of struts has a second angle between them, each pair of adjacent struts of the third row of struts has a third angle between them, and each pair of adjacent struts of the fourth row of struts has a fourth angle between them; and wherein the second and third angles are dependent on the second and third strut lengths.

Example 52. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein each strut of the first row of struts has a first length and a first width, each strut of the second row of struts has a second length and a second width, each strut of the third row of struts has a third length and a third width, and each strut of the fourth row of struts has a fourth length and a fourth width; wherein the first, second, third, and fourth lengths are each different from one another, and wherein the first, second, third, and fourth widths are each different from one another.

Example 53. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; and a valvular structure disposed within the frame, the valvular structure comprising a plurality of leaflets, each leaflet comprising a main body, an outflow edge portion, a cusp edge portion, and opposing laterally projecting tabs each having an outflow edge; wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts; wherein the outflow edge portion of each leaflet is colinear with the outflow edges of the laterally projecting tabs.

Example 54. The prosthetic heart valve of any example herein, particularly example 53, wherein each pair of adjacent struts of the first row of struts has a first angle between them, each pair of adjacent struts of the second row of struts has a second angle between them, each pair of adjacent struts of the third row of struts has a third angle between them, and each pair of adjacent struts of the fourth row of struts has a fourth angle between them; and wherein the first angle is about 120 degrees, the second angle is about 84 degrees, the third angle is about 84 degrees, and the fourth angle is about 120 degrees.

Example 55. A prosthetic heart valve, comprising: a radially expandable and compressible annular frame comprising a plurality of interconnected angled struts defining a plurality of circumferentially extending rows of cells arranged between an outflow end and an inflow end of the frame including an outflow row of cells, an intermediate row of cells, and an inflow row of cells; wherein the plurality of interconnected angled struts is arranged to form a plurality of circumferentially extending rows of struts, including a first row of struts at the outflow end of the frame, a second row of struts, one or more additional rows of struts, and a third row of struts at the inflow end of the frame, the second and one or more additional rows of struts being disposed between the first row of struts and the third row of struts; wherein each pair of adjacent struts of the first row of struts has a first angle between them, each pair of adjacent struts of the second row of struts has a second angle between them, and each pair of adjacent struts of the third row of struts has a third angle between them; wherein when the frame is in a radially expanded configuration, the struts of the second row of struts are positioned substantially parallel to an axis extending longitudinally through the frame.

Example 56. The prosthetic heart valve of any example herein, particularly example 55, wherein the diameter of the frame at the axial height of the second row of struts is constrained by the length of the second row of struts.

Example 57. The prosthetic heart valve of any example herein, particularly any one of examples 55-56, wherein when the frame is expanded to a diameter of 31 mm, the first angle is about 169 degrees, the second angle is about 163 degrees, and the third angle is about 170 degrees.

Example 58. The prosthetic heart valve of any example herein, particularly, any of examples 1-57, wherein the prosthetic heart valve is sterilized.

The features described herein with regard to any example can be combined with other features described in any one or more of the other examples, unless otherwise stated. For example, any one or more of the features of one prosthetic valve frame can be combined with any one or more features of another prosthetic valve frame.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve, comprising:
a radially expandable and compressible annular frame comprising a plurality of interconnected angled struts defining a plurality of circumferentially extending rows of cells arranged between an outflow end and an inflow end of the frame;
wherein the plurality of interconnected angled struts is arranged to form a plurality of circumferentially extending rows of struts, including an outflow row of struts at the outflow end of the frame, one or more intermediate rows of struts, and an inflow row of struts at the inflow end of the frame, the one or more intermediate rows of struts disposed between the outflow row of struts and the inflow row of struts;
wherein each strut of the outflow row of struts has a width greater than a width of each strut of the one or more intermediate rows of struts; and
wherein the plurality of circumferentially extending rows of cells comprises a first row of first cells disposed at the outflow end and at least partially defined by the first and second rows of struts, and a second row of second cells disposed adjacent to the first row of first cells and at least partially defined by the second and third rows of struts, wherein each first cell has a first axial length that is longer than a second axial length of each second cell.

2. The prosthetic heart valve of claim 1, wherein each strut of the inflow row of struts has a width greater than the width of each strut of the one or more intermediate rows of struts and less than the width of each strut of the outflow row of struts.

3. The prosthetic heart valve of claim 1, wherein the width of each strut of the outflow row of struts is between 0.32 mm and 0.44 mm.

4. The prosthetic heart valve of claim 1, wherein the width of each strut of the outflow row of struts is 0.4 mm.

5. The prosthetic heart valve of claim 1, wherein the width of each strut of the one or more intermediate rows of struts is between 0.22 mm and 0.34 mm.

6. The prosthetic heart valve of claim 1, wherein the width of each strut of the one or more intermediate rows of struts is 0.28 mm.

7. The prosthetic heart valve of claim 1, wherein the width of each strut of the inflow row of struts is between 0.3 mm and 0.42 mm.

8. The prosthetic heart valve of claim 1, wherein the width of each strut of the inflow row of struts is 0.37 mm.

9. The prosthetic heart valve of claim 1, wherein the outflow row of struts is a first row of struts, the one or more intermediate rows of struts comprises second, third, and fourth rows of struts, and the inflow row of struts is a fifth row of struts.

10. The prosthetic heart valve of claim 1, wherein the plurality of circumferentially extending rows of cells comprises four rows of cells.

11. The prosthetic heart valve of claim 1, wherein the outflow row of struts form pairs of angled struts, wherein the struts of each pair of angled struts are connected together at their adjacent ends by an outflow apex region, and wherein the outflow apex region curves between the pair of angled struts and has a narrowed width relative to each strut of the pair of angled struts.

12. The prosthetic heart valve of claim 1, wherein the frame further comprises a plurality of axially extending struts that extend between the outflow row of struts and a first row of the one or more intermediate rows of struts and define axial sides of a first row of first cells.

13. The prosthetic heart valve of claim 12, wherein a portion of the plurality of axially extending struts are axially extending window struts that define commissure windows, further comprising a plurality of leaflets secured together at their adjacent sides to form commissures, and wherein the commissures are secured to the commissure windows of the frame.

14. A prosthetic heart valve, comprising:
a radially expandable and compressible annular frame comprising a plurality of interconnected angled struts defining a plurality of circumferentially extending rows of cells arranged between a first end and a second end of the frame; and
a plurality of leaflets disposed within the frame and secured together at their adjacent sides to form commissures;
wherein the plurality of interconnected angled struts is arranged to form a plurality of circumferentially extending rows of angled struts, including a first row of angled struts at the first end of the frame, one or more intermediate rows of angled struts, and a second row of angled struts at the second end of the frame, the one or more intermediate rows of angled struts disposed between the first row of angled struts and the second row of angled struts;
wherein each strut of the first row of angled struts has a width greater than a width of each strut of the one or more intermediate rows of angled struts and each strut of the second row of angled struts;
wherein the frame further comprises a plurality of axially extending struts that extend between the first row of angled struts at the first end of the frame and a first row of the one or more intermediate rows of angled struts and define axial sides of a first row of first cells;
wherein a portion of the plurality of axially extending struts are axially extending window struts that define commissure windows; and
wherein the commissures are secured to the commissure windows of the frame.

15. The prosthetic heart valve of claim 14, wherein the width of each strut of the second row of angled struts is greater than the width of each strut of the one or more intermediate rows of angled struts.

16. The prosthetic heart valve of claim 14, wherein the width of each strut of the second row of angled struts is less than the width of each strut of the first row of angled struts.

17. The prosthetic heart valve of claim 14, wherein the width of each strut of the first row of angled struts is 0.4 mm.

18. The prosthetic heart valve of claim 14, wherein the width of each strut of the one or more intermediate rows of angled struts is 0.28 mm.

19. The prosthetic heart valve of claim 14, wherein the width of each strut of the second row of angled struts is 0.37 mm.

20. A prosthetic heart valve, comprising:
a radially expandable and compressible annular frame comprising a plurality of interconnected angled struts defining a plurality of circumferentially extending rows of cells arranged between an outflow end and an inflow end of the frame;
wherein the plurality of interconnected angled struts is arranged to form a plurality of circumferentially extending rows of struts, including a first row of struts at the outflow end of the frame, a second row of struts, a third row of struts, and a fourth row of struts at the inflow end of the frame, the second and third rows of struts disposed between the first row of struts and the fourth row of struts;
wherein each strut of the first row of struts has a first width, each strut of the second of struts has a second width, each strut of the third row of struts has a third width, and each strut of the fourth row of struts has a fourth width; and
wherein the second and third widths are different from one another.
